(19) United States
(12) Patent Application Publication
(10) Pub. No.: US 2023/0338315 A1
(43) Pub. Date: Oct. 26, 2023

(54) METHOD OF PRODUCING PHARMACEUTICAL COCRYSTALS FOR ADDITIVE MANUFACTURING

(71) Applicant: BOARD OF REGENTS, THE UNIVERSITY OF TEXAS SYSTEM, Austin, TX (US)

(72) Inventors: Mohammed MANIRUZZAMAN, Pflugerville, TX (US); Jiaxiang ZHANG, Pflugerville, TX (US); Rishi THAKKAR, Austin, TX (US)

(21) Appl. No.: 18/041,475

(22) PCT Filed: Aug. 14, 2021
(86) PCT No.: PCT/US21/46048

§ 371 Date: Feb. 13, 2023

Related U.S. Application Data
(60) Provisional application No. 63/065,810, filed on Aug. 14, 2020.

(51) Int. Cl. A61K 31/196; A61K 45/06; A61K 47/12; A61K 31/428; A61K 31/496; A61K 31/55; A61K 31/194; A61K 31/455; A61K 31/4439; A61K 31/616; A61K 31/192

(52) U.S. Cl. CPC A61K 31/196; A61K 45/06; A61K 47/12; A61K 31/428; A61K 31/496; A61K 31/55; A61K 31/194; A61K 31/455; A61K 31/4439; A61K 31/616; A61K 31/192; B33Y 70/00

(57) ABSTRACT

The present disclosure provides cocrystals of an active pharmaceutical ingredient and a co-former. The co-former may be either an excipient or a second active pharmaceutical ingredient. These particular co-crystals may be made through either an extension based process or a solvent based method. These cocrystals may be used in additive manufacturing processes. These pharmaceutical compositions may be used in the treatment of a disease or disorder.

This application claims the benefit of priority to U.S. Provisional Application No. 63/065,810, filed on Aug. 14, 2020, the entire contents of which are hereby incorporated by reference.

### BACKGROUND

### 1. Field

The present disclosure relates generally to the field of pharmaceutical cocrystals and pharmaceutical dosage forms thereof. More particularly, it concerns compositions and methods of making these cocrystals and development of dosage forms using additive manufacturing techniques thereof.

### 2. Description of Related Art

Oral drug administration of small molecular drug entities has been accepted as the most common route, which is also cost-friendly and easily accessible. However, according to the pharmaceutical reports, most active pharmaceutical ingredients (APIs) in the discovery and development pipeline have been identified as having poor water-solubility resulting in drugs that lead to poor oral bioavailability. Synthesis of cocrystals is an approach to improve the solubility of poorly soluble APIs as well as physical properties such as compactibility or tabletability of water soluble APIs. Also, the general oral dose administration takes effect slowly and is only valid for a short period. Approaches such as multiple-dose, polymeric oral-controlled dose, or osmotic pump have been tried to extend the therapeutic time, but it inevitably results in low patient compliance, slow drug release, high cost and time for development.

Nowadays, there are many approaches that have been tried in pharmaceutical drug development aiming to improve the solubility, dissolution, bioavailability, and stability of various poorly water-soluble drugs; cocrystals could be an optimal solution for such purpose. Pharmaceutical cocrystals refer to, “solids that are crystalline single-phase materials composed of two or more different crystalline compounds generally in a stoichiometric ratio which are neither solvates nor simple salts.” These cocrystals can improve the solubility of the starting compound such as the API, as well as enhancing the delivery and clinical performance of drug products by modulating drug solubility, pharmacokinetics, and bioavailability. Multidrugs could also be loaded in cocrystals for additive or synergistic treatment as well. Other than the enhancement of mechanical properties, cocrystals can be highly patentable as novel drug product intermediates.

3D printing—a layer-by-layer production of 3D objects with the help of digital designs—is also known as additive manufacturing (AM). Compared with the traditional dosage manufacturing processes, pharmaceutical 3D printing can create complex, on-demand personalized products. The pharmaceutical AM has gained increasing interest over the past few years within the pharmaceutical industry, as reflected by the increasing number of scientific publications and patents. Especially after the Food and Drug Administration (FDA) approved the first 3D-printed drug product in August 2015, the pharmaceutical AM received a boost as an emerging pharmaceutical manufacturing technique.

Therefore, there remains a need to develop one or more cocrystals that contain an active pharmaceutical ingredient and a co-former which may be used in dosage forms through additive manufacturing techniques.

### SUMMARY

The present disclosure provides pharmaceutical compositions comprising one or more cocrystals that may be prepared using solvent evaporation methods or hot melt extrusion and can be used in an additive manufacturing techniques to develop pharmaceutical dosage forms. In some embodiments, the compositions comprising a co-crystal, wherein the co-crystal comprises
(A) an active pharmaceutical ingredient; and
(B) a co-former;
   wherein the active pharmaceutical ingredient is a chemotherapeutic agent, an antibiotic, an antiviral agent, afenamic acid derivatives, lenalidomide, fleroxacin, or wherein the active pharmaceutical ingredient is ibuprofen and the co-former is saccharin, the active pharmaceutical ingredient is acetylsalicylic acid and the co-former is saccharin or the active pharmaceutical ingredient is carbamazepine and the co-former is maleic acid.

In some embodiments, the composition comprises at least 50% of the active pharmaceutical ingredient and the co-former as a cocrystal. In some embodiments, the composition comprises at least 75% of the active pharmaceutical ingredient and the co-former as a cocrystal. In some embodiments, the composition comprises at least 90% of the active pharmaceutical ingredient and the co-former as a cocrystal. In some embodiments, the composition comprises at least 95% of the active pharmaceutical ingredient and the co-former as a cocrystal. In some embodiments, the composition comprises at least 97% of the active pharmaceutical ingredient and the co-former as a cocrystal. In some embodiments, the composition comprises at least 99% of the active pharmaceutical ingredient and the co-former as a cocrystal.

In some embodiments, the active pharmaceutical ingredient is a BCS Class II drug. In other embodiments, the active pharmaceutical ingredient is a BCS Class IV drug. In some embodiments, the active pharmaceutical ingredient is an active pharmaceutical ingredient with a melting point of less than 250° C. such as less than 200° C. In some embodiments, the cocrystal has a melting point of less than 250° C. In some embodiments, the co-crystal melting point is less than 200° C.

In some embodiments, the active pharmaceutical ingredient is a chemotherapeutic agent such as lenalidomide. In other embodiments, the active pharmaceutical ingredient is the antibiotic such as fleroxacin. In other embodiments, the active pharmaceutical ingredient is a fenamic acid derivative such as mefenamic acid. In other embodiments, the active pharmaceutical ingredient is a nonsteroidal anti-inflammatory such as mefenamic acid, ibuprofen, or acetylsalicylic acid.

In some embodiments, the co-former interacts with the active pharmaceutical ingredient through one or more non-covalent interactions. In some embodiments, the non-covalent interactions are ionic interactions, hydrogen bonding, halogen bonding, van der Waals forces, π-π interactions, or hydrophobic effects. In some embodiments, the co-former and the active pharmaceutical ingredient interact with two or more non-covalent interactions. In some embodiments, the co-former is a compound which modifies the solubility of the active pharmaceutical ingredient. In some embodiments, the co-former is a compound which is sparingly soluble and modifies the solubility of the active pharmaceutical ingredient. In some embodiments, the co-former is a compound which is sensitive to the environment and modifies the solubility of the active pharmaceutical ingredient. In some embodiments, the compound is sensitive to the pH of the environment. In other embodiments, the compound is sensitive to the temperature of the environment. In some embodiments, the co-former is a compound that has no therapeutic effect.

In other embodiments, the co-former is a second active pharmaceutical ingredient. In some embodiments, the second active pharmaceutical ingredient is for the same disease or disorder as the first active pharmaceutical ingredient. In other embodiments, the second active pharmaceutical ingredient is for a different disease or disorder as the first active pharmaceutical ingredient.

In some embodiments, the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio. In some embodiments, the functional group is a NH₂, OH, C(O), C(O)OH, SH, or a nitrogen containing heterocycle. In some embodiments, the functional group is a nitrogen containing heterocycle, NH₂, OH, or SH.

In some embodiments, the co-former is a flavoring compound such assaccharin. In other embodiments, the co-former is a carboxylic acid such as maleic acid. In other embodiments, the co-former is a vitamin or a vitamin derivative such as nicotinamide. In other embodiments, the co-former is a second active pharmaceutical ingredient. In other embodiments, active pharmaceutical ingredient is ibuprofen and the co-former is saccharin. In other embodiments, the active pharmaceutical ingredient is acetylsalicylic acid and the co-former is saccharin. In other embodiments, the active pharmaceutical ingredient is carbamazepine and the co-former is maleic acid.

In some embodiments, the pKₐ of the active pharmaceutical ingredient and the pKₐ of the co-former have a pKₐ difference of less than 3. In some embodiments, the pKₐ difference is less than 2. In some embodiments, the pKₐ difference is less than 1. In some embodiments, the pKₐ difference is less than 0.5.

In some embodiments, the composition further comprises an excipient. In some embodiments, the excipient is a pharmaceutically acceptable thermoplastic polymer. In some embodiments, the active pharmaceutical ingredient or the co-former is not soluble in the pharmaceutically acceptable thermoplastic polymer. In some embodiments, the active pharmaceutical ingredient and the co-former are not soluble in the pharmaceutically acceptable thermoplastic polymer. In some embodiments, the co-crystal has been prepared using hot-melt extrusion. In other embodiments, the co-crystal has been prepared using a solvent evaporation method.

In some embodiments, the co-crystal comprises an active pharmaceutical ingredient and a co-former in a molar ratio from about 1:10 to about 10:1. In some embodiments, the molar ratio is from about 2:1 to about 1:2. In some embodiments, the molar ratio is about 2:1. In some embodiments, the molar ratio is about 1:1. In other embodiments, the molar ratio is about 1:2.

In some embodiments, the composition is deposited onto a medical device such as a tablet. In some embodiments, the tablet comprises an infill density from about 10% to about 90%. In some embodiments, the infill density is from about 20% to about 70%. In some embodiments, the infill density is from about 30% to about 50%. In some embodiments, the infill density is 30% or 50%. In some embodiments, the compositions have been recrystallized onto the medical device.

In yet another aspect, the present disclosure provides methods of preparing a composition comprising a co-crystal comprising:
(A) obtaining an active pharmaceutical ingredient and a co-former to obtain a physical mixture;
(B) subjecting the physical mixture to an extrusion process to obtain a composition comprising a co-crystal;
   wherein the extrusion process comprises two or more temperature zones.

In some embodiments, the composition comprises at least 50% of the active pharmaceutical ingredient and the co-former as a cocrystal. In some embodiments, the composition comprises at least 75% of the active pharmaceutical ingredient and the co-former as a cocrystal. In some embodiments, the composition comprises at least 90% of the active pharmaceutical ingredient and the co-former as a cocrystal. In some embodiments, the composition comprises at least 95% of the active pharmaceutical ingredient and the co-former as a cocrystal. In some embodiments, the composition comprises at least 97% of the active pharmaceutical ingredient and the co-former as a cocrystal. In some embodiments, the composition comprises at least 99% of the active pharmaceutical ingredient and the co-former as a cocrystal.

In some embodiments, the active pharmaceutical ingredient is a BCS Class II drug. In other embodiments, the active pharmaceutical ingredient is a BCS Class IV drug. In some embodiments, the active pharmaceutical ingredient is an active pharmaceutical ingredient with a melting point of less than 250° C. such as less than 200° C. In some embodiments, the cocrystal has a melting point of less than 250° C. In some embodiments, the co-crystal melting point is less than 200° C.

In some embodiments, the active pharmaceutical ingredient is a chemotherapeutic agent such as lenalidomide. In other embodiments, the active pharmaceutical ingredient is the antibiotic such as fleroxacin. In other embodiments, the active pharmaceutical ingredient is a fenamic acid derivative such as mefenamic acid. In other embodiments, the active pharmaceutical ingredient is a nonsteroidal anti-inflammatory such as mefenamic acid, ibuprofen, or acetylsalicylic acid.

In some embodiments, the co-former interacts with the active pharmaceutical ingredient through one or more non-covalent interactions. In some embodiments, the non-covalent interactions are ionic interactions, hydrogen bonding, halogen bonding, van der Waals forces, π-π interactions, or hydrophobic effects. In some embodiments, the co-former and the active pharmaceutical ingredient interact with two or more non-covalent interactions. In some embodiments, the co-former is a compound which modifies the solubility of the active pharmaceutical ingredient. In some embodiments, the co-former is a compound which is sparingly soluble and modifies the solubility of the active pharmaceutical ingredient. In some embodiments, the co-former is a compound which is sensitive to the environment and modifies the solubility of the active pharmaceutical ingredient. In some embodiments, the compound is sensitive to the pH of the environment. In other embodiments, the compound is sensitive to the temperature of the environment. In some embodiments, the co-former is a compound that has no therapeutic effect.

In other embodiments, the co-former is a second active pharmaceutical ingredient. In some embodiments, the second active pharmaceutical ingredient is for the same disease or disorder as the first active pharmaceutical ingredient. In other embodiments, the second active pharmaceutical ingredient is for a different disease or disorder as the first active pharmaceutical ingredient.

In some embodiments, the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio. In some embodiments, the functional group is a NH₂, OH, C(O), C(O)OH, SH, or a nitrogen containing heterocycle.

In some embodiments, the functional group is a nitrogen containing heterocycle, NH₂, OH, or SH.

In some embodiments, the co-former is a flavoring compound such assaccharin. In other embodiments, the co-former is a carboxylic acid such as maleic acid. In other embodiments, the co-former is a vitamin or a vitamin derivative such as nicotinamide. In other embodiments, the co-former is a second active pharmaceutical ingredient.

In some embodiments, the pKₐ of the active pharmaceutical ingredient and the pKₐ of the co-former have a pKₐ difference of less than 3. In some embodiments, the pKₐ difference is less than 2. In some embodiments, the pKₐ difference is less than 1. In some embodiments, the pKₐ difference is less than 0.5.

In some embodiments, the composition further comprises an excipient. In some embodiments, the excipient is a pharmaceutically acceptable thermoplastic polymer. In some embodiments, the active pharmaceutical ingredient or the co-former is not soluble in the pharmaceutically acceptable thermoplastic polymer. In some embodiments, the active pharmaceutical ingredient and the co-former are not soluble in the pharmaceutically acceptable thermoplastic polymer. In some embodiments, the co-crystal has been prepared using hot-melt extrusion. In other embodiments, the co-crystal has been prepared using a solvent evaporation method.

In some embodiments, the co-crystal comprises an active pharmaceutical ingredient and a co-former in a molar ratio from about 1:10 to about 10:1. In some embodiments, the molar ratio is from about 2:1 to about 1:2. In some embodiments, the molar ratio is about 2:1. In some embodiments, the molar ratio is about 1:1. In other embodiments, the molar ratio is about 1:2.

In some embodiments, each of the two or more temperature zones are each at a distinct temperature. In some embodiments, the extrusion process comprises two, three, four, five, six, seven, or eight temperature zones. In some embodiments, the extrusion process comprises three, four, or five temperature zones. In some embodiments, the extrusion process comprises four temperature zones.

In some embodiments, the first temperature zone has a temperature from about 30° C. to about 150° C. In some embodiments, the first temperature zone is from about 50° C. to about 100° C. In some embodiments, the first temperature zone is from about 60° C. to about 80° C. In some embodiments, the first temperature zone is about 70° C.

In some embodiments, the second temperature zone has a temperature from about 50° C. to about 200° C. In some embodiments, the second temperature zone is from about 75° C. to about 180° C. In some embodiments, the second temperature zone is from about 100° C. to about 160° C. In some embodiments, the second temperature zone is about 140° C.

In some embodiments, the third temperature zone has a temperature from about 75° C. to about 250° C. In some embodiments, the third temperature zone is from about 100° C. to about 220° C. In some embodiments, the third temperature zone is from about 140° C. to about 200° C. In some embodiments, the third temperature zone is about 160° C.

In some embodiments, the fourth temperature zone has a temperature from about 75° C. to about 300° C. In some embodiments, the fourth temperature zone is from about 100° C. to about 250° C. In some embodiments, the fourth temperature zone is from about 150° C. to about 225° C. In some embodiments, the fourth temperature zone is about 180° C.

In some embodiments, the ejection temperature is a temperature from about 75 ° C. to about 250° C. In some embodiments, the ejection temperature is from about 100° C. to about 220° C. In some embodiments, the ejection temperature is from about 140° C. to about 200° C. In some embodiments, the ejection temperature is about 160° C.

In some embodiments, the melting point of the pharmaceutically acceptable thermoplastic polymer is below the melting point of the active pharmaceutical ingredient or co-former. In some embodiments, the melting point of the pharmaceutically acceptable thermoplastic polymer is below the melting point of the active pharmaceutical ingredient and co-former.

In some embodiments, the extrusion process has a rotation speed from about 10 rpm to about 250 rpm. In some embodiments, the rotation speed is from about 20 rpm to about 150 rpm. In some embodiments, the rotation speed is from about 25 rpm to about 100 rpm. In some embodiments, the rotation speed is about 50 rpm.

In some embodiments, the extrusion process has a feed rate of the physical mixture from about 1 g/min to about 50 g/min. In some embodiments, the feed rate is from about 2 g/min to about 20 g/min In some embodiments, the feed rate is from about 2 g/min to about 10 g/min. In some embodiments, the feed rate is about 5 g/min

In still yet another aspect, the present disclosure provides pharmaceutical compositions prepared according to the methods described herein.

In another aspect, the present disclosure provides methods of preparing a pharmaceutical composition comprising:
(A) obtaining a composition described herein or composition prepared as described herein;
(B) subjecting the composition to an additive manufacturing technique to obtain a pharmaceutical composition;
   wherein the pharmaceutical composition is prepared as a unit dose.

In some embodiments, the additive manufacturing technique is vat photopolymerization, material jetting, binder jetting, powder bed fusion, material extrusion, directed energy deposition, or sheet lamination. In some embodiments, the additive manufacturing technique is a fused deposition modeling technique. In some embodiments, the composition is present as a filament. In some embodiments, the composition is present as a powder, granule, or a particle. In some embodiments, the composition further comprises a pharmaceutically acceptable polymer. In some embodiments, the composition is deposited onto a dosage form. In other embodiments, the additive manufacturing technique is binder spraying. In other embodiments, the additive manufacturing technique is selective laser sintering. In some embodiments, the unit dose is an oral dosage form. In some embodiments, the oral dosage form is a tablet or capsule.

In still another aspect, the present disclosure provides methods of preparing a drug-loaded medical device comprising:
(A) obtaining a composition described herein or composition prepared as described herein;
(B) dissolving the composition in a solvent to form a drug-containing solution; and
(C) placing an unloaded medical device into the drug-containing solution and allowing the composition to crystalize onto the medical device to form a drug-loaded medical device.

In some embodiments, the methods comprise heating the drug-containing solution. In some embodiments, the drug-containing solution is heated to a temperature from about 30° C. to about 150° C. In some embodiments, the temperature is from about 40° C. to about 100° C. In some embodiments, the temperature is from about 50° C. to about 80° C. In some embodiments, the solvent is an organic solvent. In some embodiments, the organic solvent is a C1-C8 alcohol such as ethanol. In some embodiments, the medical device is a tablet. In some embodiments, the table has an infill density from about 10% to about 90%. In some embodiments, the infill density is from about 20% to about 70%. In some embodiments, the infill density is from about 30% to about 50%. In some embodiments, the infill density is 30% or 50%.

In still yet another aspect, the present disclosure provides methods of treating or preventing a disease or disorder in a patient comprising administering to the patient in need thereof a therapeutically effective amount of a composition described herein or a composition prepared according to the methods described herein; wherein the active pharmaceutical ingredient is sufficient to treat or prevent the disease or disorder.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the schematic demonstration of HME and additive manufacturing platforms for personalized drug product development.

FIG. 2 shows the schematic demonstration of manufacturing novel ibuprofen-saccharin cocrystal using the HME process.

FIGS. 3A & 3B shows the hot-staged polarized light microscopy pictures of (FIG. 3A) physical mixture and (FIG. 3B) cocrystals of ibuprofen-saccharin.

FIG. 4 shows the Fourier-transform infrared spectroscopy spectrum of ibuprofen, solvent growth ibuprofen-saccharin cocrystal, HME ibuprofen-saccharin cocrystal, physical mixture, and saccharin from top to bottom.

FIG. 5 shows the Raman spectrum of ibuprofen, solvent growth ibuprofen-saccharin cocrystal, HME ibuprofen-saccharin cocrystal, physical mixture, and saccharin from top to bottom.

FIG. 6 shows the XRD spectrum of HME ibuprofen-saccharin cocrystal, saccharin, ibuprofen from top to bottom.

FIG. 7 shows the demonstration of the 3D printed pharmaceutical devices.

FIG. 8 shows the DSC of pure mefenamic acid, saccharin, cocrystals prepared by solvent evaporation and cocrystals prepared using hot melt extrusion.

FIG. 9 shows the XRD diffraction patterns of solvent evaporation of mefanamic acid-saccharin cocrystal, saccharin, and mefanamic acid from top to bottom.

FIG. 10 shows the XRD diffraction patterns of solvent evaporation of fleroxacin-saccharin cocrystal, saccharin, and fleroxacin from top to bottom

FIG. 11 shows the XRD diffraction patterns of solvent evaporation of acetylsalicylic acid-saccharin cocrystal, saccharin, and acetylsalicylic acid from top to bottom

FIG. 12 shows the XRD diffraction patterns of solvent evaporation of fleroxacin-nicotinamide cocrystal, nicotinamide, and fleroxacin from top to bottom

FIG. 13 shows the XRD diffraction patterns of solvent evaporation of carbamazepine-maleic acid (1:1) cocrystal, maleic acid (1:1), and carbamazepine from top to bottom

FIG. 14 shows the demonstration of the screw configuration used herein.

FIG. 15 shows a demonstration of the IBU, NTM, and IBU-NTM cocrystals molecules.

FIG. 16 shows the PLM figures of IBU, NTM, physical mixtures, and IBU-NTM cocrystals.

FIG. 17 shows the DSC curves of IBU, NTM, physical mixtures, and IBU-NTM cocrystals.

FIG. 18 shows the PXRD curves of IBU, NTM, physical mixtures, and IBU-NTM cocrystals.

FIG. 19 shows the FTIR spectra of IBU, NTM, physical mixtures, and IBU-NTM cocrystals.

FIG. 20 shows the Raman spectra of IBU, NTM, physical mixtures, and IBU-NTM cocrystals.

FIG. 21 shows the demonstration of the cocrystal granules obtained from 50, 75, and 150 extrusion batches.

FIG. 22 shows the demonstration of the LLD, SCH, and LLD-SCH cocrystals molecules.

FIG. 23 shows the PLM graphs of LLD, SCH, physical mixtures, and LLD-SCH cocrystals.

FIG. 24 shows the DSC curves of LLD, SCH, physical mixtures, and LLD-SCH cocrystals.

FIG. 25 shows the PXRD results of LLD, SCH, physical mixtures, and LLD-SCH cocrystals.

FIG. 26 shows the demonstration of the discharge port of extrusion process and particle size of extruded LLD-SCH cocrystals.

FIG. 27 shows the particle size and distribution of the LLD-SCH cocrystals.

FIG. 28 shows the demonstration of the extrusion dynamics, and the PLM graphs of sample collected from feeding, convey, mixing zone 5, and discharge zones.

FIG. 29 shows the demonstration of the ASA, NTM, and ASA-NTM cocrystals molecules.

FIG. 30 shows the DSC curves of ASA, NTM, physical mixtures, and ASA-NTM cocrystals.

FIG. 31 shows the PLM graphs of ASA, NTM, physical mixtures, and ASA-NTM cocrystals.

FIG. 32 shows the PXRD graphs of ASA, NTM, physical mixtures, and ASA-NTM cocrystals.

FIG. 33 shows the FTIR spectra of ASA, NTM, physical mixtures, and ASA-NTM cocrystals.

FIG. 34 shows the Raman spectra of ASA, NTM, physical mixtures, and ASA-NTM cocrystals.

FIG. 35 shows the mefenamic acid (MA)-Saccharin (SAC) interactions.

FIG. 36 shows the solvent evaporation gives phase separation and poor yield for cocrystals (Right image-samples collected from the top, left image-samples collected from the bottom).

FIG. 37 shows the melting points of drug (MA) and conformer (SAC), and heat cool heat DSC of the physical mixtures (molar ratio 1:1).

FIG. 38 shows the thermogravimetric analysis of mefenamic acid where decarboxylation begins at 180° C.

FIG. 39 shows the mass spectroscopy reveals decarboxylation of mefenamic acid on hot melt extrusion processing.

FIG. 40 shows the strategies for continuous manufacturing of cocrystals using hot melt extrusion using carrier excipients.

FIG. 41 shows the preformulation studies show that presence of Kollidon® VA64 (Blue trendline) can delay the decomposition of mefenamic acid (Orange trendline).

FIG. 42 shows the pure MA-SAC cocrystals manufactured using polymer assisted HME based continuous manufacturing platform, with extrudates suitable for 3D printing.

FIG. 43 shows the Dissolution test performed at pH 6.8 depicts a fourfold increase in the solubility of mefenamic acid produced using continuous, solvent-free, hot melt extrusion, no degradation or decarboxylation of mefenamic acid was observed in the extruded samples.

FIG. 44 shows the demonstration of the blank FDM printed tablets and the IBU-NTM cocrystals loaded tablets.

FIG. 45 shows the demonstration of the direct compressed physical mixture tablets and the IBU-NTM cocrystals loaded SLS tablets sintered at 1 mm/s and 10 mm/s.

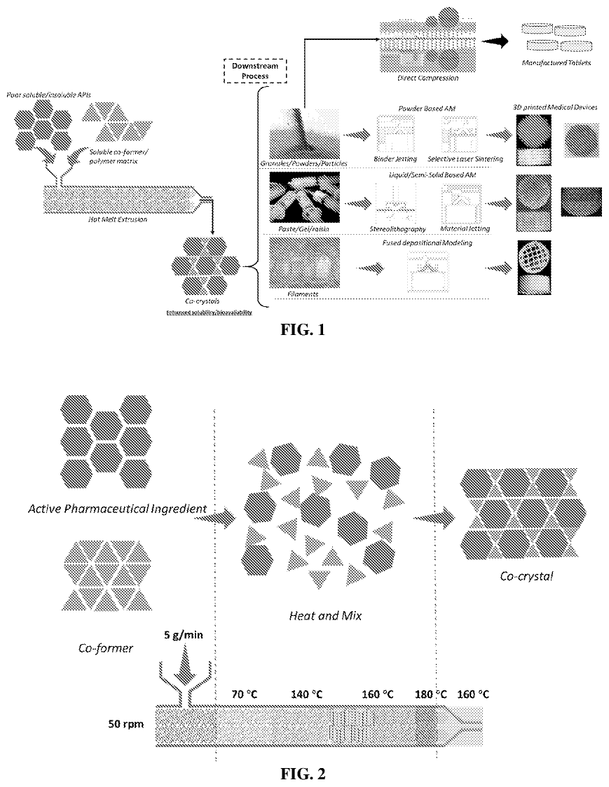

### DESCRIPTION OF ILLUSTRATIVE EMBODIMENTS

In some aspects, the present disclosure relates to the methods of preparing a cocrystal of the active pharmaceutical ingredient and a conformer, for particular co-crystals of antibiotics, anti-virals, and cancer therapeutics. The cocrystal can be prepared using a variety of different methods including solvent evaporation or hot melt extrusion. In one particular embodiment, these co-crystals may be manufactured using solid state hot melt extrusion. These co-crystals may then be utilized in the additive manufacturing proceses.

In some embodiments, the pharmaceutical cocrystals described herein are manufactured using a twin-screw corotating extrusion platform, including one or more gravimetric feeders, a hermetic closure barrel with temperature control, and a proper die for shaping the extruded cocrystals. The manufactured cocrystals with proper physicochemical properties may be used in conventional drug production platforms such as tableting and capsule filling, without a downstream process, or subjected to the additive manufacturing platforms such as fused depositional modeling as well as selective laser sintering, and binder spraying. In other aspects, the pharmaceutical cocrystals described herein may be subjected to a downstream process such as milling and mixing with other excipients for AM platforms. These and more details are described below.

### I. Co-Crystals

In some embodiments, the co-crystals described herein are comprised of an active pharmaceutical ingredient and one or more co-formers. The co-formers may be either a non-therapeutic excipient such as a flavoring agent, an acid, or a vitamin or in the alternative, the co-former is a second therapeutic agent for either the same disease or a different disease. These two compounds may be present in the co-crystals in a molar ratio from about 10:1 to about 1:10, from about 5:1 to about 1:5, or from about 2:1 to about 1:2. The molar ratio is from about 10:1, 8:1, 6:1, 4:1, 3:1, 2:1, 1:1, 1:2, 1:3, 1:4, 1:6, 1:8, to about 1:10, or any range derivable therein. The composition described herein may comprise at least 50%, at least 75%, at leas 90%, at least 92.5%, at least 95%, at least 98%, at least 99% (w/w) of the active pharmaceutical ingredient and the co-former are present as the co-crystal. The co-crystal may have a melting point of less than 500° C., less than 400° C., less than 300° C., less than 250° C., less than 200° C., or less than 150° C.

### A. Active Pharmaceutical Ingredient

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the cells lining the gastrointerstinal tract. According to the BCS, drug substances are classified as follows: Class I—High Permeability, High Solubility; Class II—High Permeability, Low Solubility; Class III—Low Permeability, High Solubility; and Class IV—Low Permeability, Low Solubility.

While the pharmaceutical compositions and methods described herein can be applied to any BCS class of drugs, BCS class II and IV are of interest for the pharmaceutical compositions described herein. Additionally, other API that are of specific consideration are those that are high melting point drugs such as a drug that has a melting point of greater than 200° C. Alternatively, the API used herein may have a melting point from about 25° C. to about 1,000° C., from about 100° C. to about 750° C., or from about 200° C. to about 500° C. In particular, the melting point may be greater than 200° C., 250° C., 300° C., 400° C., 500° C., 300° C., 700° C., 750° C., 800° C., 900° C., or 1,000° C.

In some embodiments, the active pharmaceutical ingredient in these compositions is a non-steroidal anti-inflammatory compound such as acetylsalicylic acid, diflunisal, salicylic acid and its salts, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, phenylbutazone, anthranilic acid derivatives (fenamates), mefenamic acid, meclofenamic acid, flufenamic acid, tolfenamic acid, celecoxib, parecoxib, lumiracoxib, etoricoxib, firocoxib, sulfonanilides, nimesulide, clonixin, licofelone, or h-harpagide.

In other embodiments, the active pharmaceutical ingredient is an antimicrobial agent such as antibiotic or anti-viral agent. Some non-limiting examples of antimicrobial agents include bactericidal antibiotics include penicillin, cephalosporin, polymyxin, rifamycin, lipiarmycin, quinolones, and sulfonamides or bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the antimicrobial agent is a drug which acts against mycobacteria such as cycloserine, capreomycin, ethionamide, rifampicin, rifabutin, rifapentine, and streptomycin. Other antibiotics include arsphenamine, chloramphenicol, fosfomycin, fusidic acid, metronidazole, mupirocin, platensimycin, quinupristin, dalfopristin, thiamphenicol, tigecycline, tinidazole, or trimethoprim. In other embodiments, the antimicrobial agents is an antiviral agent such as abacavir, aciclovir, acyclovir, adefovir, amantadine, amprenavir, ampligen, arbidol, atazanavir, atripla, balavir, boceprevirertet, cidofovir, combivir, dolutegravir, daruavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, interferon type I, type II, and type III, lamivudine, lopinavir, loviride, maraviroc, moroxydine, methisazone, nelfinavir, nevirapine, nexavir, oseltamivir, penciclovir, peramivir, pleconaril, podophyllotoxin, raltegravir, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, traporved, valaciclovir, valganciclovir, vicriviroc, vidarabine, viramidine, zalcitabine, zanamivir, or zidovudine. In some embodiments, the antiviral agents is an anti-retroviral, a fusion inhibitor, an integrase inhibitor, an interferon, a nucleoside analogues, a protease inhibitor, a reverse transcriptase inhibitor, a synergistic enhancer, or a natural product such as tea tree oil.

In other embodiments, the active pharmaceutical ingredient is a chemotherapeutic agent. Some non-limiting examples of chemotherapeutic agents include include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin η^{I} and calicheamicin toil; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-1-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone;

vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

### B. Co-Former

In some aspects, the present disclosure comprises one or more excipients formulated into pharmaceutical compositions as co-former with the active pharmaceutical ingredient to form a co-crystal. The co-former may be an excipient such as pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients that may be used in the co-crystals include vitamins, polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the compositions are substantially, essentially, or entirely free of any other excipient other than the co-former. In other embodiments, the composition comprises one or more excipients. In other embodiments, the co-crystals comprise a co-former which is a second active pharmaceutical ingredient rather than an excipient.

The co-former may interact with the active pharmaceutical ingredient though one or more non-covalent interactions. These non-covalent interactions may include ionic interactions, hydrogen bonding, halogen bonding, van der Waals forces, π-π interactions, or hydrophobic effects. The interactions between the co-formers and active pharmaceutical ingredients may comprise two, three, four, five, or six non-covalent interactions which may be the same or a different type of non-covalent interaction. The co-former may interact with the active pharmaceutical ingredients in such a way that it modifies the properties of the active pharmaceutical ingredient including changing its solubility profile. The co-former itself may be sparingly soluble, sensitive to the environment such as the pH or the temperature.

### C. Further Excipients

In some aspects, the present disclosure comprises one or more excipients formulated into pharmaceutical compositions with the co-crystals such as a pharmaceutically acceptable thermoplastic polymer. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients include polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient.

In some aspects, the pharmaceutical composition may further comprise one or more inorganic or organic material that may be used to bulk up a composition to obtain an effective amount of the compound. The filler may be an inert inorganic or organic compound such as a salt like a calcium, magnesium, sodium, or potassium salt or a sulfate, chloride, or nitrate salt. Commonly used organic compounds include carbohydrates, sugars, and sugar derivatives such as mannitol, lactose, starch, or cellulose.

Furthermore, the pharmaceutical compositions described herein have a concentration of filler ranging from about 1% to about 99% w/w. In some embodiments, the amount of each absorbent is from about 1% to about 99% w/w, from about 25% to about 98% w/w, 50% to about 98% w/w, or 75% to about 97% w/w. The amount of each absorbent may be from about 10%, 25%, 30%, 35%, 40%, 45%, 50%, 55%, 60%, 65%, 70%, 75%, 80%, 85%, 30 90%, 92.5%, 95%, 96%, 97%, 98%, to about 99%, or any range derivable therein. In some embodiments, the pharmaceutical composition is substantially, essentially, or entirely free of any other fillers.

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active pharmaceutical ingredient in a given solution so that the particles formed contain more than one active pharmaceutical ingredient.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the pharmaceutical composition to enhance the efficacy and efficiency of the active pharmaceutical ingredient in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include sucrose, trehalose, golden sheen, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidones, and tyloxapol.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing agent, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferably, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzyme inhibitors. Particular examples are such enzyme inhibitors include but are not limited to inhibitors that inhibit cytochrome P-450 enzyme and inhibitors that inhibit monoamine oxidase enzyme. Bioavailability can be indicated by the C. or the AUC of the active pharmaceutical ingredient as determined during in vivo testing, where C. is the highest reached blood level concentration of the active pharmaceutical ingredient over time of monitoring and AUC is the area under the plasma-time curve. Enhanced bioavailability can be represented as the ratio of C. or the AUC of the active pharmaceutical ingredient in a pharmaceutical composition of the present disclosure compared to C. or the AUC of the reference standard the active pharmaceutical ingredient under the same conditions. This C. or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In some aspects, the amount of the excipient in the pharmaceutical composition is from about 0.5% to about 99% w/w, from about 1% to about 95% w/w, from about 10% to about 95% w/w, or from about 20% to about 80% w/w. The amount of the excipient in the pharmaceutical composition comprises from about 0.5%, 1%, 2.5%, 5%, 7.5%, 10%, 15%, 20%, 25%, 30%, 35%, 40%, 45%, 50%, 55%, 60%, 65%, 70%, 75%, 80%, 82.5%, 85%, 87.5%, 90%, 91%, 92%, 93%, 94%, 95%, 96%, 97%, 98%, to about 99% w/w, or any range derivable therein, of the total pharmaceutical composition. In one embodiment, the amount of the excipient in the pharmaceutical composition is at 50% to 90% w/w of the total weight of the pharmaceutical composition.

### II. Pharmaceutical Compositions

In some aspects, the present disclosure provides pharmaceutical compositions containing an active pharmaceutical ingredient or a pharmaceutically acceptable salt, ester, derivative, analog, pro-drug, or solvates thereof and a co-former which may be an excipient or a second active pharmaceutical ingredient as a co-crystal. These compositions may be used to prepare a pharmaceutical composition from a starting material such as a filament or powder that exhibits one or more favorable properties such as exhibiting a free-flowing property as an angle of repose, sufficient strength, sufficient stress, bend angle, diameter, viscosity, or Carr's Index. The co-crystal and the active pharmaceutical ingredient may comprise the active pharmaceutical ingredient and the co-former in a molar ratio from about 0.1 to about 10, from about 0.25 to about 4, or from about 0.5 to about 2. The molar ratio of the active pharmaceutical ingredient and the co-former is from about 0.1, 0.2, 0.25, 0.33, 0.5, 1, 2, 3, 4, 5, or 10. Alternatively, these molar ratios may be further described as from about 1:10 to about 10:1, from about 1:4 to about 4:1, or from about 1:2 to about 2:1. The molar ratio of the active pharmaceutical ingredient and the co-former is from about 1:10, 1:5, 1:4, 1:3, 1:2, 1:1, 2:1, 3:1, 4:1, 5:1, or 10:1. The amound of the composition which contains the co-crystal is greater than about 50%, 60%, 70%, 80%, 90%, 95%, 96%, 97%, 98%, or 99%. The pharmaceutical composition comprises from about 50%, 60%, 65%, 70%, 75%, 80%, 85%, 90%, 92.5%, 95%, 96%, 97%, 97.5%, 98%, 98.5%, 99%, 99.5%, to about 99.9%, or any range derivable therein of the co-crystal. The co-crystals may be formed between an active pharmaceutical ingredient and either an excipient or a second active pharmaceutical ingredient. These components of the co-crystals may have a pKₐ difference of less than 3, less than 2, less than 1.5, less than 1, less than 0.75, less than 0.5, or less than 0.25.

These compositions may exhibit one or more free-flowing properties such as having a flowability as measured by fixed funnel method with the angle of repose of less than 25°. These compositions may exhibit a flowability as measured by the angle of repose of less than about 25, less than about 27.5, less than about 30, less than about 32.5, less than about 35, less than about 37.5, or less than about 40. The flowability may be from about 25 to about 40, or from about 25 to about 30. The flowability may be from about 2, 4, 5, 6, 8, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or any range derivable therein. The flowability of the pharmaceutical composition is measured by fixed funnel method. The simplest method for the determination of the angle of repose is the “poured” angle. A funnel with a wide outlet is affixed at a distance of 10 cm above the bench, where a piece of paper is placed directly beneath the funnel. The granules are added while the funnel is closed. The contents flow through and collect on the paper. The diameter of the cone (D) and two opposite sides (l₁+l₂) are measured with rulers. The angle of repose (θ) is calculated from the equation arc cos[D/(l₁+l₂)]. The relationship between flow properties and angle of repose has been established. When the angle of repose is less than 25 degrees, the flow is said to be excellent; on the other hand, if the angle of repose is more than 40 degrees, the flow is considered to be poor. These pharmaceutical compositions may be present as agglomerations and used in either a batch, semi-continuous, continuous manufacturing process. The active pharmaceutical ingredient may act as a binder between the absorbent particles within the pharmaceutical composition.

In other aspects, the present pharmaceutical compositions may exhibit a mean or average particle size distribution greater than 25 μm, greater than 50 μm, or greater than 60 μm. In some embodiments, the pharmaceutical compositions exhibit a mean or average particle size from about 25 μm to about 500 μm, 30 μm to about 400 μm, 35 μm to about 350 μm, 40 μm to about 300 μm, 50 μm to about 250 μm, 50 μm to about 200 μm, 50 μm to about 150 μm, 55 μm to about 125 μm, or from about 60 μm to about 100 μm. The mean or average particle size of the pharmaceutical composition comprises from about 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 150 μm, 175 μm, 200 μm, 250 μm, 300 μm, 350 μm, 400 μm, 450 μm, 500 μm, 600 μm, 700 μm, 800 μm, 900 μm, to about 1000 μm, or any range derivable therein. The mean or average particle size of the pharmaceutical composition may be determined by mesh analysis using a sonic sifter. The particle size distribution of the dried granules can also be determined by a dry laser diffraction technique or scanning electron microscopy. In the mesh shake method, US #16 to 400 may be used for sieve analysis and sieve are kept in ascending order on each other. 15-30 g sample is weighted and put into the #16 mesh (smallest #mesh were used as the top one). The shakers were set at 60 shake/sec for 30 min (or until no powders pass through the sieve), and record the weight in each mesh and then calculate the size and distribution. Alternatively, the dry particle laser diffraction characterization methods were used to determine the particle size and distribution. A laser diffractometer with a disperser with the detection range from 0.1-875 μm was used to collect the particle size and distribution data. An optimal concentration of 0.1% was setup as the trigger condition and a feed rate of 50% and 3 bar. Finally, SEM pictures may be used. In this methodology, the SEM pictures are taken and particles size were determined using a 3 points method.

Similarly, these compositions may exhibit a particle diameter, D₅₀, wherein 50% of the particles in the composition are larger than this particular particle size. The composition may have a D₅₀ of less than 100 μm, less than 75 μm, less than 60 μm, or less than 50 μm. Additionally, the composition may exhibit a particle diameter, D₉₀, wherein 90% of the particles in the composition are smaller than this particular particle size. The particles may have a D₉₀ wherein the D₉₀ is greater than 25 μm, greater than 40 μm, or greater than 50 μm. Alternatively, the D₉₀ may be less than 100 μm, less than 90 μm, less than 80 μm, or less than 75 μm. In some embodiments, the D₉₀ may be from about 10 μm to about 150 μm, from about 25 μm to about 100 μm, from about 50 μm to about 80 μm. The D₉₀ may be from about 10 μm, 25 μm, 30 μm, 40 μm, 45 μm, 50 μm, 55 μm, 60 tm, 65 μm, 70 μm, 75 μm, 80 μm, 85 μm, 90 μm, 10 μm, 100 μm, 105 μm, 110 μm, 120 μm, to about 125 μm, or any range derivable therein. The dry particle laser diffraction characterization methods were used to determine the particle size and distribution. A laser diffractometer with a disperser with the detection range from 0.1-875 μm was used to collect the particle size and distribution data. An optimal concentration of 0.1% was setup as the trigger condition and a feed rate of 50% and 3 bar.

In some aspects, the pharmaceutical composition may exhibit a Carr's Index is from about 5 to about 28, from about 5 to about 25, from about 5 to about 21, from about 5 to about 15, or from about 15 to about 25. The Carr's Index may be from about 2, 4, 5, 6, 7, 8, 9, 10, 11, 12, 13, 14, 15, 16, 17, 18, 19, 20, 21, 22, 23, 24, 25, 28, 30, 32, 35, 38, to about 40, or any range derivable therein. Carr's Index of the pharmaceutical composition may be determined by tapped density which is measured after a powder sample is subjected to mechanically tapping. The measurement procedure for bulk density and tapped density can be found in the US Pharmacopeia. Bulk density and tapped density can be used to calculate the Carr's compressibility index and Hausner ratio, which are measures of the propensity of a powder to flow and be compressed:

$$Compressibiliy index ( \% ) = \frac{tapped density - bulk density}{tapped density} \times 100 \\ Hausner ’ s ratio = \frac{Tapped density}{bulk density}$$

The bulk density of the composition may be less than 5 g/cm³, less than 4 g/cm³, less than 3 g/cm³, less than 2.5 g/cm³, less than 2.25 g/cm³, less than 2 g/cm³, less than 1.75 g/cm³, or less than 1.5 g/cm³. The bulk density may be in a range from about 0.25 g/cm³, 0.5 g/cm³, 0.75 g/cm³, 1 g/cm³, 1.25 g/cm³, 1.5 g/cm³, 1.75 g/cm³, 2 g/cm³, 2.25 g/cm³, 2.5 g/cm³, 3 g/cm³, 3.5 g/cm³, 4 g/cm³, 4.5 g/cm³, to about 5 g/cm³, or any range derivable therein. The bulk density were measured using a graduate cylinder by gently pass a quantity of powder sufficient to complete the test through a U.S. standard sieve #18 or smaller. The agglomerates formed during storage were break up before test. Approximately 100 g±1.0% (RSD) of the test sample (m) weighed were passed to a dry graduated cylinder of 250 ml (readable to 2 ml) without compacting, and read the unsettled apparent volume (V₀) to the nearest graduated unit. Calculate the bulk density in (g/cm³) using the formula m/V₀. The tapped density is measured by mechanically tapping a graduated measuring cylinder containing the powder sample. Powder samples were proceeded to a 250 ml graduated cylinder (readable to 2 ml) and a settling apparatus capable of producing 250±15 taps/min, and bulk volume (V₀) was determined using abovementioned methods. 10, 500 and 1250 taps on the same powder sample were conducted and the corresponding volumes V10, V500 and V1250 were recorded. (If the difference between V500 and V1250 is less than or equal to 2 ml, V1250 is the tapped volume. If the difference between V500 and V1250 exceeds 2 ml, repeat in increments such as 1250 taps, until the difference between succeeding measurements is less than or equal to 2 ml.) Calculate the tapped density (g/cm³) using the formula m/V_{f} in which V_{f} is the final tapped volume.

In some aspects, the present pharmaceutical composition may be exhibit compressibility that makes the composition useful for the production of pharmaceutical dosage forms such as oral forms like capsules or tablets. The pharmaceutical composition may also be used in a powder-based additive manufacturing application such as vat photopolymerization, material jetting, binder jetting, powder-bed fusion, material extrusion, directed energy deposition, or sheet lamination like fused deposition modeling, binder spraying, or selective laser sintering. These 3D printing platforms may be used in pharmaceutical manufacturing and patient-specific personalized therapy to produce on-demand pharmaceutical compositions.

### III. Manufacturing Methods

In some embodiments, the co-crystals described herein may be prepared though a solvent based method such slow evaporation, slow cooling, vapor diffusion, or liquid-liquid diffusion. Alternatively, the co-crystals may be made through a solvent free method such as extrusion.

### A. Extrusion

Thus, in one aspect, the present disclosure provides pharmaceutical compositions which may be prepared using a thermal or fusion-based high energy process. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007, DiNunzio et al., 2010a, DiNunzio et al., 2010b, DiNunzio et al., 2010c, DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher, such as a minilab compounder, or Leistritz, such as a twin-screw extruder. Alternatively, a fusion-based high energy process instrument that does not require external heat input, including such as a thermokinetic mixer as described in U.S. Pat. No. 8,486,423 and 9,339,440 may be used to process the pharmaceutical composition.

In some aspects, the extruder may comprise heating the composition through two or more temperature zones. The methods may comprise one, two, three, four, five, six, seven, or eight temperature zones. Each of these temperature zones may have a distinct temperature. The highest temperature of the temperature zones is below, at, or above the melting temperature of the co-crystal or any excipients such as a pharmaceutically acceptable polymer. The first temperature in the first temperature zone may be a temperature from about 25° C. to about 250° C. In some embodiments, the first temperature is from about 30° C. to about 150° C., from about 50° C. to about 100° C., or from about 60° C. to about 80° C. The first temperature that may be used is from about 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 140° C., 150° C., 160° C., 170° C., 180° C., 190° C., 200° C., 210° C., 220° C., 225° C., 230° C., 240° C., to about 250° C. or any range derivable therein.

The second temperature in the second temperature zone may be a second temperature from about 25 ° C. to about 300 ° C. In some embodiments, the second temperature is from about 50° C. to about 200° C., from about 75° C. to about 180° C., or from about 100° C. to about 160° C. The second temperature that may be used is from about 25° C., 30° C., 40° C., 50° C., 60° C., 70° C., 80° C., 90° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 210° C., 220° C., 225° C., 230° C., 240° C., 250° C., 260° C., 270° C., 280° C., 290° C., to about 300° C. or any range derivable therein.

The third temperature in the third temperature zone may be a third temperature from about 75° C. to about 250° C. In some embodiments, the third temperature is from about 75° C. to about 250° C., from about 100° C. to about 220° C., or from about 140 ° C. to about 200° C. The third temperature that may be used is from about 25° C., 30° C., 40° C., 50° C., 60° C., 70° C., 80° C., 90° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., to about 300° C. or any range derivable therein.

The fourth temperature in the fourth temperature zone may be a fourth temperature from about 75° C. to about 300° C. In some embodiments, the fourth temperature is from about 75° C. to about 300° C., from about 100° C. to about 250° C., or from about 150° C. to about 225° C. The fourth temperature that may be used is from about 25° C., 30° C., 40 ° C., 50° C., 60° C., 70° C., 80° C., 90° C., 100° C., 105° C., 110° C., 115° C., 120° C., 125° C., 130° C., 135° C., 140° C., 145° C., 150° C., 155° C., 160 ° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., to about 300° C. or any range derivable therein.

The fifth temperature in the fifth temperature zone may be a fifth temperature from about 75° C. to about 300° C. In some embodiments, the fifth temperature is from about 75° C. to about 300° C., from about 100° C. to about 250° C., or from about 150° C. to about 225° C. The fifth temperature that may be used is from about 25° C., 30° C., 40° C., 50° C., 60° C., 70° C., 80° C., 90° C., 100° C., 105 ° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., to about 300° C. or any range derivable therein.

The sixth temperature in the sixth temperature zone may be a sixth temperature from about 75° C. to about 300° C. In some embodiments, the sixth temperature is from about 75° C. to about 300° C., from about 100° C. to about 250° C., or from about 150° C. to about 225° C. The sixth temperature that may be used is from about 25° C., 30° C., 40° C., 50° C., 60° C., 70° C., 80° C., 90° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., to about 300° C. or any range derivable therein.

The seventh temperature in the seventh temperature zone may be a seventh temperature from about 75° C. to about 300° C. In some embodiments, the seventh temperature is from about 75° C. to about 300° C., from about 100° C. to about 250° C., or from about 150° C. to about 225° C. The seventh temperature that may be used is from about 25° C., 30° C., 40° C., 50° C., 60° C., 70° C., 80° C., 90° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., to about 300° C. or any range derivable therein.

The eighth temperature in the eighth temperature zone may be a eighth temperature from about 75° C. to about 300° C. In some embodiments, the eighth temperature is from about 75° C. to about 300° C., from about 100° C. to about 250° C., or from about 150 ° C. to about 225° C. The eighth temperature that may be used is from about 25° C., 30° C., 40 ° C., 50° C., 60° C., 70° C., 80° C., 90° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., to about 300° C. or any range derivable therein.

Finally, the co-crystals may be ejected from the device with an ejection temperature that is from from about 75° C. to about 250° C. In some embodiments, the fourth temperature is from about 75° C. to about 250° C., from about 100° C. to about 220° C., or from about 140° C. to about 200° C. The fourth temperature that may be used is from about 25° C., 30° C., 40° C., 50° C., 60° C., 70° C., 80° C., 90° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., to about 300° C. or any range derivable therein.

The extrusion methods described herein may convey material through the extruder at a feed rate from about 1 g/min to about 50 g/min The feed rate is from about 1 g/min to about 50 g/min, from about 2 g/min to about 20 g/min, or from about 2 g/min to about 10 g/min The feed rate that may be used is from about 0.5 g/min, 1 g/min, 2 g/min, 3 g/min, 4 g/min, 5 g/min, 6 g/min, 7 g/min, 8 g/min, 9 g/min, 10 g/min, 12 g/min, 14 g/min, 15 g/min, 16 g/min, 18 g/min, 20 g/min, 25 g/min, 30 g/min, 35 g/min, 40 g/min, 45 g/min, to about 50 g/min, or any range derivable therein. These methods may further comprise the use of a rotating screw that has a rotation speed from about 10 rpm to about 250 rpm. The rotation speed is from about 10 rpm to about 250 rpm, from about 20 rpm to about 150 rpm, or from about 25 rpm to about 100 rpm. The rotation speed may be from about 5 rpm, 10 rpm, 15 rpm, 20 rpm, 25 rpm, 30 rpm, 35 rpm, 40 rpm, 45 rpm, 50 rpm, 55 rpm, 60 rpm, 65 rpm, 70 rpm, 75 rpm, 80 rpm, 85 rpm, 90 rpm, 95 rpm, 100 rpm, 110 rpm, 120 rpm, 130 rpm, 140 rpm, 150 rpm, 160 rpm, 180 rpm, 200 rpm, 220 rpm, 225 rpm, 240 rpm, to about 250 rpm, or any range derivable therein.

Furthermore, the extrusion methods may further comprise using an extruder with a specific screw configuration. The extrusion methods described herein may use a screw length/diameter (L/D) ratio of greater than 10, greater than 20, specifically 25 or 40.

The screw configuration could consist of a combination of convey and kneading elements. The kneading elements L/D ratio could be 1, 1.1, 1.2, 1.3, 1.4, 1.5, 1.6, 1.7, 1.8, 1.9, 2, 2.2, 2.4, 2.6, 2.8, 3, 4, 5, 6, 7, 8, 9, or 10 or be any range derivable therein. The kneading zone could be 30°, 60°, or 90°.

### IV. Processing into Final Dosage Form

In some embodiments, the co-crystals described herein may be subjected to one or more further processing techniques to obtain a final dosage form. These methods may involve grinding or milling the pharmaceutical composition and then compressing the co-crystals into a dosage form. Following any necessary drying steps, the resulting co-crystal is milled or ground to granules or a dry powder. The granules or dry powder may then be compacted. Compacting refers to a process whereby a powder mass comprising the granules or powder is densified under high pressure in order to obtain a compact with low porosity, e.g. a tablet. In some embodiments, the composition is formulated in a manner which is amenable to oral administration. Compression of the powder mass is usually done in a tablet press, more specifically in a steel die between two moving punches. Alternatively, these methods may include the use of additive manufacturing techniques to process these compositions into their final dosage form.

### A. Additive Manufacturing Platforms

In some aspects, the pharmaceutical compositions described herein are processed in a final dosage form. The granules that are produced by the process may be further processed into a capsule or a tablet. Before formulation into a capsule or tablet, the granule may be further milled before being compressed into the capsule or tablet.

In other aspects, the pharmaceutical compositions described herein may also be used in an additive manufacturing platform. Some of the additive manufacturing platforms that may be used herein include 3D printing such as selective laser sintering or selective laser melting. Alternatively, a method such as stereolithography or fused deposition modeling may be used to obtain the final pharmaceutical composition. The pharmaceutical compositions described herein may be used these processes and exhibit a flowability with an angle of repose less than 25°. The pharmaceutical composition may have a flowability of less than 25, less than 26, less than 27, less than 28, less than 29, less than 30, less than 32.5, less than 35, or less than 40.

These pharmaceutical compositions may be processed through laser sintering wherein a laser is aimed at a specific point on the pharmaceutical composition such that material is bound together to create a solid form. The laser is passed over the surface in a sufficient amount of time and sufficient location to produce the desired dosage form. The method relates to the use of the laser-based upon the power of the laser such as the peak laser power rather than the laser duration. The method often will make use of a pulsed laser. The laser used in these methods often is a high-power laser such as a carbon dioxide laser. The process builds up the dosage form using cross-sections of the material through multiple scanning passes over the material. Additionally, the chamber of the 3D printer device may also be preheated to a temperature just below the melting point of the pharmaceutical composition such as the melting point of the composition as a whole or the active agent, the absorbent, or the surfactant. Furthermore, the method may be used without the need for a secondary feeder of material into the chamber of the device.

In some embodiments, the additive manufacturing techniques used in the present methods may include selective laser sintering 3D printing. This method may comprise use of a laser onto a composition that has been deposited into a chamber at particular locations. The laser acts to sinter the composition into a pharmaceutical composition. The formation of the final product is based upon the energy of the laser as well as the properties of the composition and the temperature of the composition and the chamber that the compositions are deposited into.

In the first part of the selective laser sintering process, the composition is deposited onto a surface in the chamber. The deposition of the composition may result in a layer, wherein the layer of the composition has a layer thickness (LT) from about 0.1 μm to about 100 mm, from about 1 μm to about 100 mm, from about 10 um to about 100 mm, from about 50 μm to about 10 mm, from about 50 μm to about 1 mm, or from about 50 μm to about 100 μm. The layer thickness may be from about 0.1 μm, 1 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 um, 145 μm, 150 μm, 175 μm, 200 μm, 250 μm, 300 μm, 350 μm, 400 μm, 450 μm, 500 μm, 600 μm, 700 μm, 750 μm, 800 μm, 900 μm, 1 mm, 5 mm, 10 mm, 25 mm, 50 mm, 75 mm, to about 100 mm

The composition deposited into the surface in the chamber may be heated to a temperature, known as the surface temperature. This surface temperature may be used to provide additional energy to the composition to assist the preparation of the final pharmaceutical composition. The surface temperature may be a temperature from about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500 ° C., or any range derivable.

Furthermore, the chamber may also be heated to a temperature known as the chamber temperature. The chamber temperature may be a temperature from about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable. In some embodiments, the chamber temperature is at least 1° C., at least 5° C., at least 10° C., at least 15° C., at least 20° C., at least 25° C., or at least 50° C. less than the surface temperature. The chamber temperature may be from 1° C. to about 50° C., 5° C. to about 25° C., 10° C. to about 25° C., or 10° C. to about 20° C. less than the surface temperature.

Once the composition has been deposited therein, the composition is exposed to a laser to sinter the composition to obtain the final pharmaceutical composition. The parameters of the laser may be used in obtaining a pharmaceutical composition with a co-crystal from the composition deposited in the chamber. The particular laser used by the process may further comprise a laser power from about 0.1 mW to about 25 W, from about 0.5 mW to about 10 W, from about 1 mW to about 1 W, or from about 1 mW to about 10 mW. The laser used herein may have a laser power from about 10 mW, 50 mW, 100 mW, 200 mW, 300 mW, 400 mW, 500 mW, 600 mW, 700 mW, 800 mW, 900 mW, 1 W, 5 W, 15 W, 20 W, to about 25 W, or any range derivable therein. The particular laser used may include a high power laser such as carbon dioxide laser, lamp or diode, pumped ND:YAG laser, and disk or fiber lasers.

In some embodiment, a 2.3 watt solid diode 455 nm wavelength (visible light, bright blue) laser may be used. The laser used may emit light with a wavelength from about 50 nm to about 15,000 nm, from about 200 nm to about 11,000 nm, or from about 200 nm to about 1,000 nm. The wavelength may be 50 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1,000 nm, 1,025 nm, 1,050 nm, 1,075 nm, 1,100 nm, 1,500 nm, 2,000 nm, 2,500 nm, 3,000 nm, 3,500 nm, 4,000 nm, 4,500 nm, 5,000 nm, 5,500 nm, 6,000 nm, 6,500 nm, 7,000 nm, 7,500 nm, 8,000 nm, 8,500 nm, 9,000 nm, 9,500 nm, 10,000 nm, 10,500 nm, 11,000 nm, 12,000 nm, 13,000 nm, 14,000 nm, to about 15,000 nm, or any range derivable therein. Furthermore, the laser used may have a specific beam size that indicates the size of the laser that strikes any particular point of the composition at a given time. The methods may further comprise using a laser with a beam size from about 0.1 μm to about 10 mm, from about 0.25 μm to about 1 mm, from about 1 um to about 500 μm, or from about 2.5 μm to about 100 μm. The beam size may be a size from about 0.1 μm, 0.5 μm, 1 μm, 2.5 μm, 5 μm, 7.5 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 250 μm, 500 μm, 750 μm, 1 mm, to about 5 mm, or any range derivable therein.

The laser may be used to sinter the composition in a pattern. During the sintering process, the laser traces a pattern over the composition to prepare the final pharmaceutical composition. The pattern is prepared by passing the laser over the composition at a specific speed known as the laser speed (LS). The laser speed may be from about 1 mm/s to about 100,000 mm/s, from about 5 mm/s to about 50,000 mm/s, from about 10 mm/s to about 1,000 mm/s, or from about 25 mm/s to about 250 mm/s. The laser speed may be from about 0.1 mm/s, 0.25 mm/s, 0.5 mm/s, 0.75 mm/s, 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein. Furthermore, the laser may pass in a pattern over the composition in the surface of the chamber. The distances between the lines in the laser's pass are known as hatches. The distance between each successive laser pass is known as the hatch spacing. The methods used herein may include using a hatch spacing from about 0.1 mm to about 250 mm, from about 0.5 mm to about 200 nm, from about 1 mm to about 150 mm, or to about 10 to about 40 mm The hatch spacing may be from about 0.1 mm, 0.25 mm, 0.5 mm, 0 75 mm, 1 mm, 5 mm, 10 mm, 11 mm, 12 mm, 13 mm, 14 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, 100 mm, 125 mm, 150 mm, 175 mm, 200 mm, 225 mm, to about 250 mm, or any range derivable therein.

Finally, the combination of the chamber temperature and the surface temperature may be used to combine with the laser energy to provide sufficient energy to obtain the pharmaceutical composition. The amount of energy that the laser imparts into the pharmaceutical composition is calculated as the electron laser density. Electron laser density may be calculated using the following formula:

$$Electron Laser Density ( \frac{J}{{mm}^{3}} ) = \frac{Laser Power ( w )}{LS \times HS \times LT}$$

The electron laser density may be an amount of energy imparted from the laser from about 1 J/mm³ to about 500 J/mm³, from about 2.5 J/mm³ to about 500 J/mm³, from about 5 J/mm³ to about 250 J/mm³, from about 7.5 J/mm³ to about 100 J/mm³, or from about 7.5 J/mm³ to about 50 J/mm³. The electron laser density is from about 1 J/mm³, 1.5 J/mm³, 2 J/mm³, 2.5 J/mm³, 3 J/mm³, 3.5 J/mm³, 4 J/mm³, 4.5 J/mm³, 5 J/mm³, 5.5 J/mm³, 6 J/mm³, 6.5 J/mm³, 7 J/mm³, 7.5 J/mm³, 8 J/mm³, 8.5 J/mm³, 9 J/mm³, 9.5 J/mm³, 10 J/mm³, 12.5 J/mm³, 15 J/mm³, 17.5 J/mm³, 20 J/mm³, 25 J/mm³, 50 J/mm³, 75 J/mm³, 100 J/mm³, 150 J/mm³, 200 J/mm³, 250 J/mm³, 300 J/mm³, 400 J/mm³, to about 500 J/mm³, or any range derivable therein.

In other embodiments, the compositions may be prepared using a fusion deposition modeling method. Fusion deposition modeling uses a filament which is then heated and extruded layer by layer upon a surface until the layers build the desired objects. Such deposition is produced from the extrusion by passing a nozzle from the extruder over the surface such that it deposits the pharmaceutical compositions onto a surface in both the x, y, and z dimensions. As the first layer is deposited, the layer may be given some time to cool before the next layer is deposited. As described above for the laser sintering methods, the extruder may be passed over the composition and the filament or other material is deposited with a specific hatch spacing. The methods used herein may include using a hatch spacing from about 5 mm to about 100 mm, from about 10 mm to about 75 nm, from about 10 mm to about 50 mm, or to about 10 to about 40 mm The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37 5 mm, 40 mm, 45 mm, 50 mm, 60 mm, 70 mm, 80 mm, 90 mm, to about 100 mm, or any range derivable therein. Each of these passes over the object may be performed at a specific speed known as the print speed. The print speed may be from about 1 mm/s to about 100,000 mm/s, from about 5 mm/s to about 50,000 mm/s, from about 10 mm/s to about 1,000 mm/s, or from about 25 mm/s to about 250 mm/s. The print speed may be from about 0.1 mm/s, 0.25 mm/s, 0.5 mm/s, 0.75 mm/s, 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein.

The platform temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable. In some embodiments, the methods contemplate a second platform temperature may be a temperature from about −125° C., −100° C., −90° C., −80° C., −75° C., −70° C., −60° C., −50° C., −40° C., −30° C., −25° C., −20° C., −10° C., 0° C., 10° C., 15° C., 20° C., 22.5° C., to about 25° C., or any range derivable.

This method can comprise the use of a filament. The filament has a diameter from about 0.5 mm to about 10 mm, from about 1 mm to about 7.5 mm, from about 1.5 mm to about 5 mm The diameter of these filaments may be from about 0.25 mm, 0 5 mm, 0.75 mm, 1 mm, 1.25 mm, 1.5 mm, 1.75 mm, 2 mm, 2.25 mm, 2.5 mm, 3 mm, 3.5 mm, 4 mm, 4.5 mm, 5 mm, 6 mm, 7 mm, 7 5 mm, 8 mm, 9 mm, to about 10 mm, or any range derivable therein. The filaments that are used in these methods may exhibit some preferred strength or stress properties. Flexibility, brittleness and stiffness properties of the filaments were evaluated to represent the printability of the filaments. For Flexibility and brittleness evaluation, filament samples were cut into 50 mm in length. A TA-XT2 texture analyzer (Texture Technologies Corp, New York, USA) with a TA-95N 3-point bend apparatus were used to test the brittleness of the extruded filaments. 25 mm supporting gape and 1 mm blade were used, and blades moving speed is 10 mm/s until reach 15mm below the samples. Each single formulation filaments were repeated 10 times. Breaking distance and load force/stress data were collected and analyzed. For stiffness analysis, filaments samples were placed on the solid platform and were cut into 5 mm in depth of the samples. The blade will cut into the sample for 35% shape change, and breaking stress/force data were collected. Each single formulation filaments were repeated 10 times. In particular, the filament may have a strength such that the forced needed to break the filament is greater than 1000 g, 2000 g, or 3000 g. Similarly, the filament may have a strength such that the forced needed to cut the filament is greater than 100 g, 200 g, or 300 g. Additionally, the stress needed to break the filament is greater than 5,000 g/mm², greater than 10,000 g/mm², or greater than 15,000 g/mm². Finally, the filaments used may have a bend angle such that the force needed to break the filament is greater than 10°, greater than 20° C., or greater than 30°. In addition, the force needed to cut into the filaments is greater than 1000 g, 2000 g, or 3000 g.

In other embodiments, the composition may be prepared using a binder spraying method. Using a binder spraying method, the method comprises applying a powder to a surface and then applying a liquid binder material to the powder such that the deposition of the next layer of powder sticks to the lower layer of powder. These methods similar to the selective laser sintering and fusion deposition methods requires the use of hatch spacing print speed. he methods used herein may include using a hatch spacing from about 5 mm to about 100 mm, from about 10 mm to about 75 nm, from about 10 mm to about 50 mm, or to about 10 to about 40 mm The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, to about 100 mm, or any range derivable therein. Each of these passes over the object may be performed at a specific speed known as the print speed. The print speed may be from about 1 mm/s to about 100,000 mm/s, from about 5 mm/s to about 50,000 mm/s, from about 10 mm/s to about 1,000 mm/s, or from about 25 mm/s to about 250 mm/s. The print speed may be from about 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein. These methods require the use of a liquid binder material. Some non-limiting examples of liquid binder materials include hydrocarbons such as: n-pentane, n-hexane, n-heptane, n-octane, n-nonane, n-decane, benzene, toluene, 2,2,4-trimethyl pentane, cyclohexane, 2,2,4-trimethylpentane, cyclohexane, ethylbenzene, ketones, acetone, methyl ethyl ketone, methyl isobutyl ketone, cyclohexanone, n-methyl-2-pyrrolidone, acetophenone; alcohols such as: methanol, ethanol, n-propanol, propanol, n-butanol, i-butanol, 2-butanol, n-amyl alcohol, i-amyl alcohol, cyclohexanol, n-octanol, ethanediol, diethylene glycol, 1,2-propanedioi; ethers such as: diethyl ether, diisopropyl ether, dibutyl ether, methyl tert-butyl ether, 1,4-dioxane, tetrahydrofuran, esters, methyl acetate, ethyl acetate, isopropyl acetate, n-butyl acetate, cellosolve acetate, glycol ethers, propylene glycol methyl ether, ethylene glycol methyl ether, ethylene glycol ethyl ether, ethylene glycol monobutyl ether; chlorinated solvents such as: methylene chloride, chloroform, carbon tetrachloride, 1,2-dichloroethane, 1, i,1-trichloroethane, trichloroethylene, perchloroethylene, monochlorobenzene, miscellaneous solvents, dimethylformamide, dimethylacetamide, dimethylsulphoxide, sulfolane, carbon disulphide, acetic acid, aniline, nitrobenzene, morpholine, pyridine, 2-nitropropane, acetonitrile, furfuraldehyde, or phenol. These materials are often characterized by their viscosity wherein the liquid binder materials may have a viscosity from 0.1 mPa*s to 250 mPa*s, from about 0.25 mPa*s to 150 mPa*s, or from about 0.5 mPa*s to about 50 mPa*s. The viscosity of the liquid binder material may be from about 0.1 mPa*s, 0.25 mPa*s, 0.5 mPa*s, 1 mPa*s, 2.5 mPa*s, 5 mPa*s, 10 mPa*s, 25 mPa*s, 50 mPa*s, 75 mPa*s, 100 mPa*s, 125 mPa*s, 150 mPa*s, 175 mPa*s, 200 mPa*s, 225 mPa*s, to about 250 mPa*s, or any range derivable therein.

### III. Definitions

The use of the word “a” or “an”, when used in conjunction with the term “comprising” in the claims and/or the specification, may mean “one,” but it is also consistent with the meaning of “one or more,” “at least one,” and “one or more than one.” As used herein “another” may mean at least a second or more.

As used herein, the terms “active pharmaceutical ingredient”, “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, to alleviate signs or symptoms of the disease. Desirable effects of treatment include decreasing the rate of disease progression, ameliorating or palliating the disease state, and remission or improved prognosis. Alleviation can occur before signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of the cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active pharmaceutical ingredient in the composition is measured by powder x-ray diffraction.

A “poorly soluble drug” refers to a drug that meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified components has been purposefully formulated into a composition and/or is present only as a contaminant or in trace amounts. The total amount of all containments, by-products, and other material is present in that composition in an amount of less than 2%. The term “essentially free of” or “essentially free” is used to represent that the composition contains less than 1% of the specific component. The term “entirely free of” or “entirely free” contains less than 0.1% of the specific component.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the thermally conductive excipient are adjacent to another atom of the thermally conductive excipient.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C. unless otherwise noted. An elevated temperature is a temperature that is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active pharmaceutical ingredient to a patient in a single administration. Such unit dose formulations that may be used include but are not limited to a single tablet, capsule, or other oral formulations, or a single vial with a syringeable liquid or other injectable formulations. The resulting product can then undergo further downstream processing to create an intermediate product, such as granules, that can then be further formulated into a unit dose such as one prepared for oral delivery as tablets, capsules, three-dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Preparation of Formulations

### Example A Formulation

The formulation was prepared by physically mixing ibuprofen (API, powders) with saccharin (co-former, powders) use a pestle and mortar.

TABLE 1
Formulation Composition

| Formulation | Function | Melting point | Molar Ratio |
|---|---|---|---|
| Ibuprofen | API | 80.8° C. | 1:1 |
| Saccharin | Conformer | 230.8° C. | |

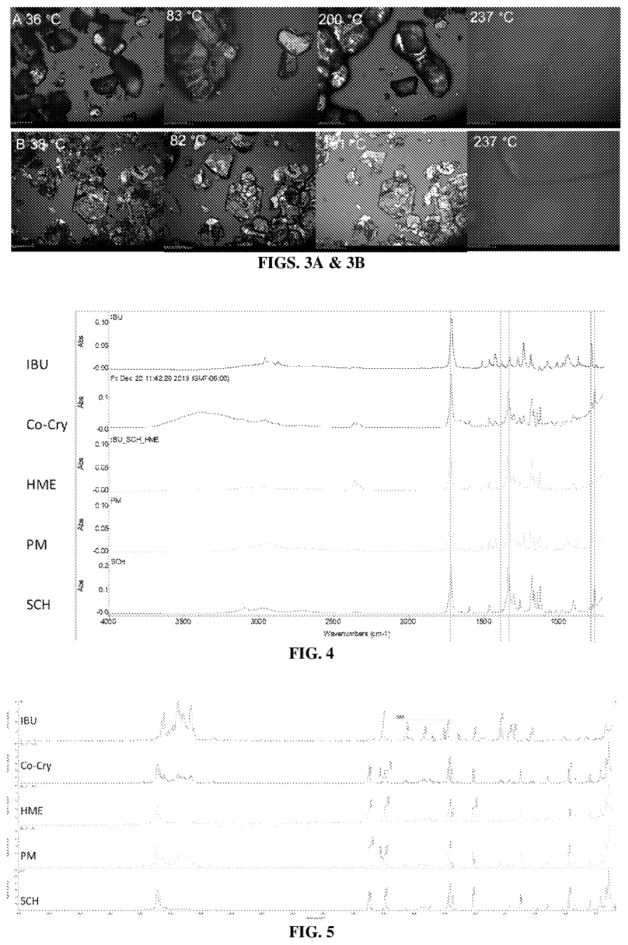

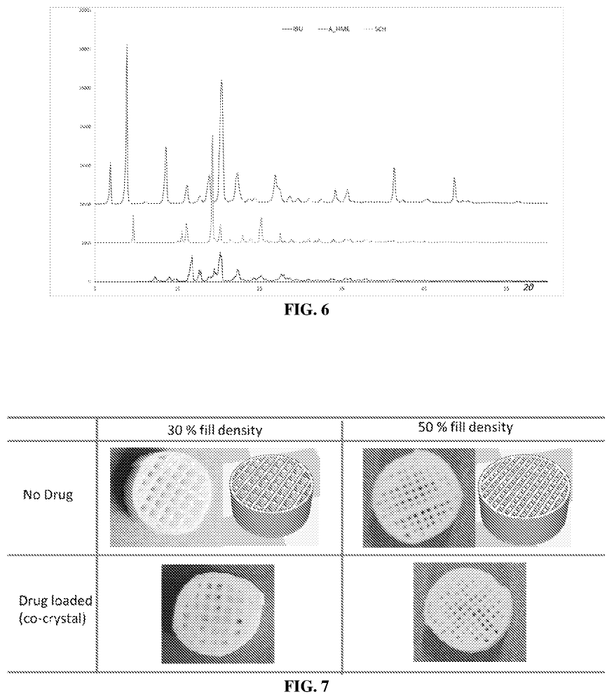

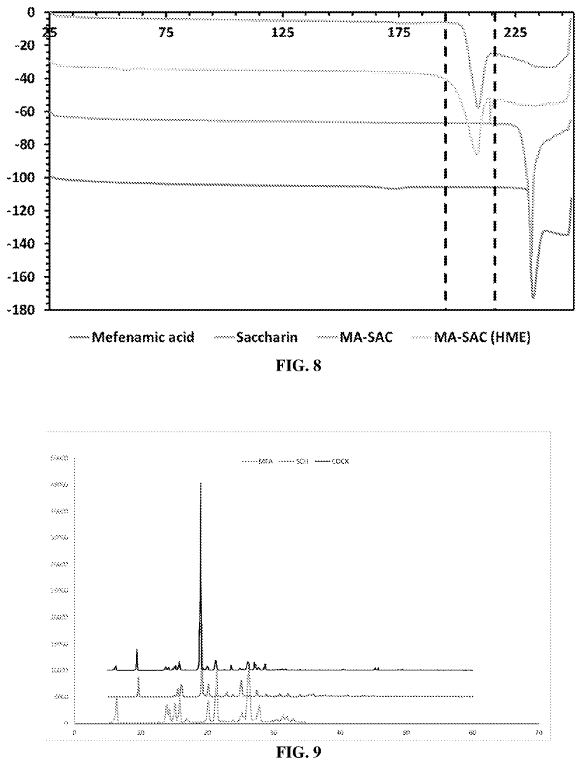

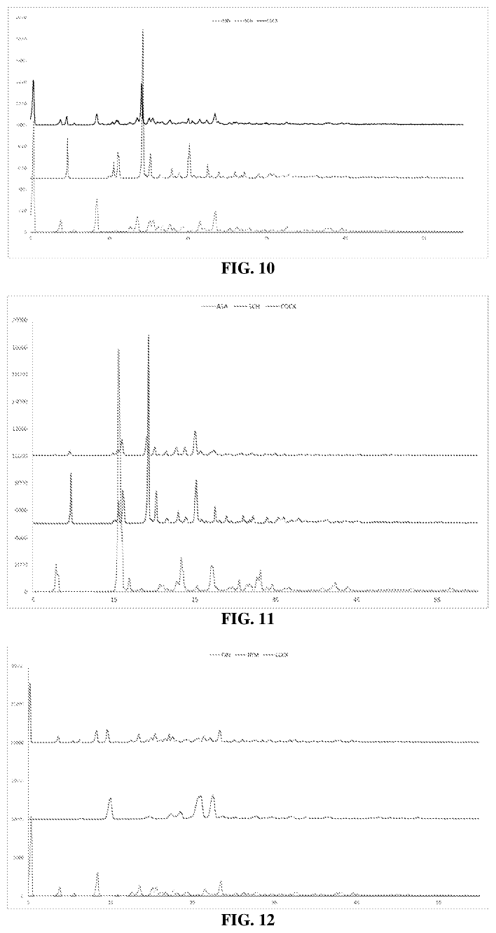

The physical mixture was fed into a Leistritz 16 mm nano extruder with four zones. One 8 L/D kneading zone were used, and processing conditions maintained in the four zones are explained in the schematic diagram (See FIG. 2). The ibuprofen-saccharin cocrystals made by solvent evaporation methods were prepared for reference purposes.

i. Characterization of the Cocrystals

The produced granules were collected and characterized using various characterization techniques.

a. DSC

8-12 mg of each the ibuprofen, saccharin, physical mixture, HME ibuprofen-saccharin cocrystal, and solvent growth ibuprofen-saccharin cocrystal were subjected to the DSC analysis, which the samples were ramped from 25° C. to 240° C. at 10° C./min. The curve indicates that the formation of the ibuprofen-saccharin cocrystal because the identical melting peak around 220° C. in both HME ibuprofen-saccharin cocrystal and solvent growth ibuprofen-saccharin cocrystal. However, there are also ibuprofen melting peaks shown in the cocrystal samples, which might because the HME process was not optimized which results in the incomplete formation of cocrystals.

b. Polarized Light Hot Stage Microscopy

Samples of the ibuprofen-saccharin physical mixture and ibuprofen-saccharin cocrystal were subjected to the polarized light hot stage microscope which can visually identify the crystal melting characteristics. As shown in FIG. 3, the ibuprofen melts at around 80° C. while the saccharin maintained its solid form until 237° C., which indicates there are two crystalline structures in the mixture, and they have limited interactions. On the contrary, the majority of the ibuprofen-saccharin cocrystal showed melting around 220° C., however, there are small amounts of ibuprofen not forming cocrystals exists.

c. Fourier-Transform Infrared Spectroscopy

Samples of the ibuprofen-saccharin physical mixture and ibuprofen-saccharin cocrystal were subjected to Fourier-transform infrared spectroscopy which can identify the functional groups and determine the non-covalent interactions in the molecular level. The identical peaks marked in FIG. 4 indicates there are no chemical reactions between ibuprofen and saccharin molecular, that is ibuprofen within co-crystalline structure still shown the same pharmacological effects as ibuprofen crystals. Also, the intensified peak ranged from 3000-3500 cm⁻¹ and 2300-2400 cm⁻¹ indicates the interaction between the carboxyl and alcoholic hydroxyl groups, which were resulted in the formation of the hydrogen bonds.

d. Raman Spectroscopy

Samples of the ibuprofen-saccharin physical mixture and ibuprofen-saccharin cocrystal were subjected to Raman spectroscopy which can identify the chemical bond movements and determine the non-covalent bond formation in the molecular level. The identical peaks marked in FIG. 5 indicates there are no chemical reactions between ibuprofen and saccharin molecular, that is ibuprofen within co-crystalline structure still shown the same pharmacological effects as ibuprofen crystals. Also, the attenuated peak ranged from 2950-3100 cm⁻¹ indicates the weekend vibration frequency of carboxyl groups, which were resulted in the formation of the hydrogen bonds.

### Example 2—Additive Manufacturing to Create Cocrystal Loaded Pharmaceutical Dosage Forms

The manufacturing process can be classified as described herein that the drug-free medical devices were printed and submerged in the solution with cocrystals manufactured using HME for drug loading. The medical devices that designed in a cylinder shape with 10 mm in diameter, 4.5 mm in height, and 30% and 50% infill density were prepared by fused depositional modeling additive manufacturing platform. The print parameters are listed in table 2. An excessive amount of cocrystals were added into the ethanol at room temperature, then heated to 60° C. to dissolve. Then the printed devices were submerged into the solution while cooling down, where the cocrystals recrystallized inside the porous structures of the printed devices (FIG. 7). The examples showed in FIG. 7, where the drug loading of the 30% porosity tablets was 3.8% w/w while drug ratio was 6.7% w/w in 50% porosity tablets. In general, the tablets can be designed with 10%400% infill density, where the drug loads could be 0-90% w/w.

TABLE 2
Printing parameters for the medical devices

| Layer height | 0.1 mm |
|---|---|
| Wall thickness | 0.4 mm |
| Top/bottom thickness | 0 |

| | | |
|---|---|---|
| Infill density | 30% | 50% |

| | |
|---|---|
| Infill pattern | Lines |
| Printing temperature | 180° C. |
| Build plate temperature | 50° C. |
| Print speed | 60 mm/s |

### Example 3—Additional Co-Crystal Formulations

### Example B-J

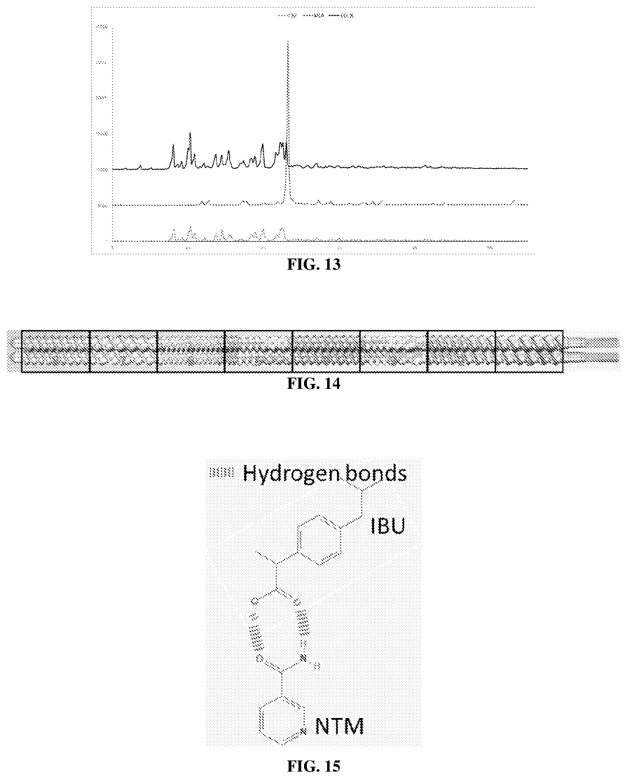

A list of additional co-crystal formulations prepared using HME platform is listed in table 3 below. Such API-co-former combinations were validated by widely available solid states analysis (data were not shown, similar to example A) that the cocrystals were obtained. The listed cocrystals have similar application paths with example A, which can be used for widely available additive manufacturing platforms including FDM 3D printing. The DSC characterization of the mefenamic acid/saccharin formulation is shown in FIG. 8. The XRD diffraction patterns of these additional cocrystal compositions are shown in FIGS. 9-13.

TABLE 3
Additional cocrystal compositions
using solid-state HME processing

| | | API:Co-former |
|---|---|---|
| API | Co-former | ratio |
| Mefenamic acid | Saccharin | 1:1 |
| Fleroxacin | Saccharin | 1:1 |
| Acetylsalicylic acid | Saccharin | 1:1 |
| Fleroxacin | Nicotinamide | 1:1 |
| Carbamazepine | Maleic acid | 1:1 |
| Carbamazepine | Maleic acid | 2:1 |
| Ibuprofen (IBU) | Nicotinamide (NTM) | 1:1 |
| Lenalidomide (LLD) | Saccharin (SCH) | 1:1 |
| Acetylsalicylic acid (ASA) | Nicotinamide (NTM) | 1:1 |

### Preparation of Examples B-J

A Leistritz ZSE 12 HP-PH 12 mm twin screw corotating extruder with eight individual zones was used to extrude all drug-coformer combinations in order to produce cocrystals. The extrusion was performed at different temperature profiles depending on different formulations. There are several process parameters that significantly affect the formation of the cocrystals such as processing temperature, degree of mixing/kneading, screw-speed, and residence time. In this work, the twin screw configuration was designed identical, where two isolated kneading zones were set at zone 3 and 5, while the other zones are either convey, vent, or discharge zone FIG. 14.

### A. Screw Configuration and Temperature Profiles

The process temperature or temperature profiles were the primary factors that affect the formation of the cocrystals. In the current work, the materials are fed into the extruder at ambient temperature (zone 1) and then conveyed (zone 2) to the first mixing zone (zone 3) to assure a thorough mixing of two ingredients. Then the drug-coformer compositions are conveyed (zone 4) to the second mixing zone (zone 5), where the temperature was set to melt one or both ingredients to ensure a homogenous mixing of the components at the molecular level. Then conveying and venting (zone 6) let any condensed moisture to evaporate (if any) out of the barrel, and then convey zone 7 and 8 cool down the molten extrudates to ambient temperature before materials are discharged from the barrel. So, the setting temperature of the mixing zone 5 is critical to the continuous extrusion of the cocrystals. With the help of the mechanical energy inputs, the mixing zone 5 temperature was set at either lower or between the melting points of the two ingredients (drug-coformer). Detailed temperature set up for each formulation is summarized in Table 4 below.

TABLE 4
Temperature setting for different formulations

| | Zone | | | | | | | |
|---|---|---|---|---|---|---|---|---|
| Formulation | Zone 8 | Zone 7 | Zone 6 | Zone 5 | Zone 4 | Zone 3 | Zone 2 | Zone 1 |
| 1-IBU-NTM | 25 | 35 | 55 | 80 | 70 | 55 | 30 | 25 |
| 2-LLD-SCH | 92 | 119 | 170 | 190 | 170 | 120 | 75 | 54 |
| 3-ASA- | 50 | 75 | 100 | 130 | 100 | 75 | — | — |
| NTM | | | | | | | | |
| 4-MA-SAC | 70 | 90 | 150 | 170 | 170 | 150 | 40 | 25 |

### B. Feeding Rates and Screw Speed

For formulation ASA-NTM combinations, screw speed and feeding rate were studied. Three extrusion conditions were tried at the same temperature settings where feeding-screw speed was set at 6 g/min-50 rpm, 9 g/min-75 rpm, and 18 g/min-150 rpm.

### C. Process Quality

i. Reproducibility

Reproducibility studies were conducted with formulation ASA-NTM, where the 6 g/min-50 rpm condition was repeated 3 times.

ii. Process Dynamic

Process dynamic studies were conducted with formulation 2 LLD-SCH, where the screw was pulled out from the barrel while pausing the extrusion in the middle, and samples were collected from the screws.

iii. Measurement of the Residence Time

Residence times were recorded during the extrusion process, reaching the steady states, where the torque changes within 5% variations. Around 100 mg of tracer dye (Rhodamine B) were added in zone 1 during the extrusion, and the time observed color change at zone 8 was recorded as the residence time.

### Characterization of Cocrystals

### A. DSC

A DSC Q20 equipment (TA® instruments, Delaware, USA) was used for the DSC analysis. Approximately, 5-10 mg of pure API, co-former, physical mixtures, and extruded cocrystals were sealed in the standard aluminum pan and lids and ramped from 25 to complete melting temperature (depending on the samples) at a rate of 20° C./min. In all DSC experiments, ultra-purified nitrogen was used as the purge gas at a 50 mL/min flow rate. The data were collected and plotted as a plot of temperature (° C.) versus reverse heat flow (mW) using Excel software (Version 2007).

### B. Powder X-ray Diffraction (PXRD)

The solid state of pure API, co-former, physical mixtures, and extruded cocrystals were investigated using PXRD analysis using a benchtop PXRD instrument (MiniFlex, Rigaku Corporation, Tokyo, Japan). Briefly, the samples were scanned from a 2θ angle of 5 to 60 degrees, with a scan speed of 2 degrees/minute, scan step of 0.02 degrees, and the resultant scan resolution of 0.0025. The voltage was set at 45V, and the current was set at 15 mA during the scan process. The data were collected and plotted as a stacked plot of 2θ versus intensity using Excel software (Version 2007).

### C. Hot-Stage Polarized Light Microscopy (PLM)

An Olympus BX53 polarizing photomicroscope (Olympus America Inc., Webster, TX, USA) equipped with Bertrand Lens was used to analyze the crystallinity of the pure API, co-former, physical mixtures, and extruded cocrystals. The samples were spread out evenly onto a glass slide. A coverslip was used to press and spread the samples as monolayer particles. The slide was placed on the microscope stage. All samples were observed under 10× magnification for birefringence property in crystalline substances. A QICAM Fast 1394 digital camera (Qlmaging, BC, Canada) with a 530 nm compensator (U-TP530, Olympus® corporation, Shinjuku City, Tokyo, Japan) was used to capture the images.

### D. Fourier-Transform Infrared (FTIR) Spectroscopy

The pure API, co-former, physical mixtures, and extruded cocrystals were investigated using an Antaris analyzer with Nicolet iS50 series spectrophotometer (Thermal Fisher Scientific, Madison, WI, USA). Samples were positioned onto the face of the diamond crystal of the ATR unit, and the tip of the micrometer clamp was compressed onto the particles to allow adequate contact to get a characteristic spectrum. Backgrounds were collected using 32 scans for each 4 h during the FTIR measurements, while the spectrum of the sample was by scanning the specimens 32 times over a 750-4000 cm⁻¹ range at a resolution of 4 cm⁻¹ per sample.

### E. Raman Spectroscopy

The pure API, co-former, physical mixtures, and extruded cocrystals were investigated using an Antaris analyzer (Thermal Fisher Scientific, Madison, WI, USA) equipped with a Raman scanning unit. Samples were positioned onto a 9-well sample holder. The samples spectrum was by scanning the specimens 32 times over a 300-4000 cm⁻¹ range at a 4 cm⁻¹ per sample.

### F. Cocrystals Particle Size and Distribution

Images of the cocrystals were taken using Dino-Lite optical microscopy. Particle size was measured using DinoCapture 2.0 (version 1.5.43), where particles were circled using 3-points circles. Approximately, 5 different figures for replicates of each cocrystal were taken, and diameter d was recorded then exported to Excel for further analysis.

### G. Angle of Repose (AOR)

The material was poured through a funnel to form a cone. Pouring was stopped when the pile reaches a predetermined height or the base a predetermined width. The AOR was calculated by dividing the cone height by half the width of the base of the cone. The inverse tangent of this ratio is the angle of repose.

### H. Tapped Density

Tapped density of cocrystals was obtained as the ratio of the mass of the cocrystals to the volume occupied by the powder after it has been tapped for a defined period. The tapping period is defined as the volume changing <5%, and for all the formulations, it's around 100 taps.

### 3D Printing Tablets with Cocrystals

IBU-NTM cocrystals were used as an example to conduct additive manufacturing studies.

### A. FDM

An Ultimaker S3 FDM printer was used to print 3D tablets while the tablets were then submerged into the molten ASA-NTM extrudates for drug loading. Tablets were designed as a cylinder shape, and 10 mm in diameter and 3.5 mm in thickness. Layer thickness is 0.1 mm, and the infill density is 75%.

### B. SLS

A Sintratec SLS printer was used to sintering the tablets. 5% w/w of IBU-NTM cocrystals were mixed with 90% of Soluplus and 5% golden sheen. SLS printing were performed in two conditions, where laser scanning speed were set at 1 and 10 mm/s, respectively. The hatching space were set at 12 um for both batches. tablets were designed in same cylinder shape as FDM printing. Physical mixture tablets were directly compressed as references.

### Results and Discussions

### A. Ibuprofen-Nicotinamide

The IBU-NTM were premixed and fed into the extruder at room temperature while completely melting at zone 5, and particles or granules were discharged from zone 8. As shown in FIG. 15, the IBU and NTM can form cocrystals by forming a hydrogen bond between the amine and carboxyl groups, confirmed via solid states analysis.

i. Identification

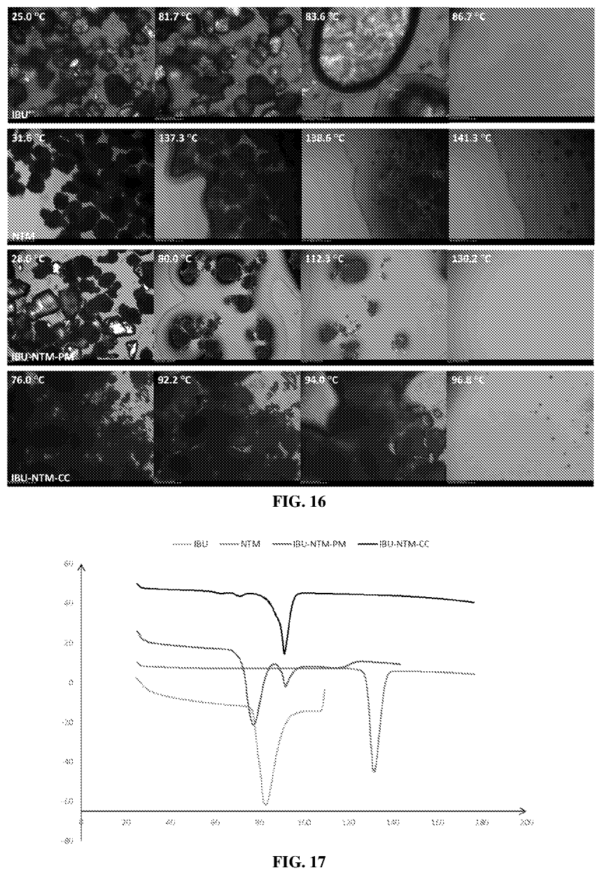

PLM figures (FIG. 16) showed that IBU and NTM melt at around 84° C. and 139° C., respectively. The physical mixture of the IBU-NTM starts melting around 80° C., which is because of IBU, while NTM melts or dissolve into the molten IBU before reaching 130° C. This is mainly because of the interaction between the two molecules' function groups, which results in the adequate miscibility of two ingredients. A small fraction starts to melt at around 90° C., which is mainly due to the existence of free IBU or smaller particles. The cocrystals showed a single step of melting at 94° C. instead of two steps shown in the physical mixtures, which potentially proved the formation of a single phase crystalline entity such as cocrystals.

DSC figures (FIG. 17) can cross-verify the observation from the PLM figures, where IBU and NTM have a melting peak of around 82.87° C. and 131.34° C., respectively. And two isolated melting peaks can be observed in the physical mixture curve, where the first peak corresponds to the melting of IBU and the second one indicates the melting of NTM. The cocrystals thermal transition showed a smaller peak around 80° C. which might be due to the presence of a very small fraction of unprocessed IBU or impurity , while the cocrystal has a melting peak of 91.29° C.

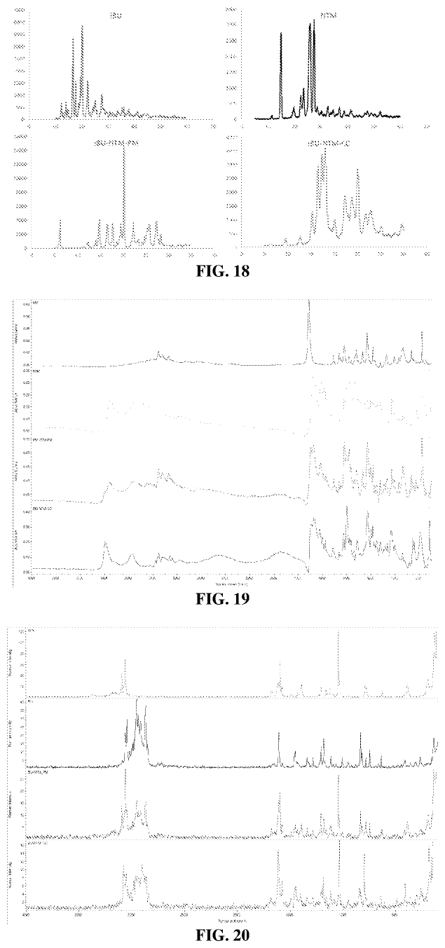

PXRD (FIG. 18) also proved the formation of the IBU-NTM cocrystals where the IBU showed characteristic peaks around 2θ of 16.80, 17.68, 19.48, 20.24, 22.32, and 27.68°, and NTM showed peaks around 2θ of 15.00, 22.30, 23.12, and 27.50°. The cocrystals showed characteristic peaks around 2θ of 16.50, 17.36, 18.10, 25.12, and 28.12°. An additional new peak at ˜10 2θ position is evident for the cocrystal formulation. This particular peak is not present in any of the bulk components nor in the physical mixture which indicates the formation of new crystal forms other than the IBU or NTM.

FTIR (FIG. 19) showed the intermolecular interactions of the cocrystals. The carboxyl group of IBU can be identified around wavenumbers of 1718 and 930 cm⁻¹, while the amine group of NTM can be identified around wavenumbers of 1540-1450 cm⁻¹. The existence of the hydrogen bond in cocrystals results in the shift of the abovementioned peaks to the lower wavenumbers.

Raman (FIG. 20) showed the intramolecular movements of the cocrystals. The N—H rocking movement of amine can be identified at the Raman shift of 1049 cm⁻¹ in NTM and physical mixtures, while it shifts to lower wavenumbers (1035 cm⁻¹) in the IBU-NTM cocrystals. The asymmetrical stretching can be identified around 3035-3110 cm⁻¹ in IBU, NTM, and physical mixtures, while it is broadened to 3003-3129 cm⁻¹ in the IBU-NTM cocrystals because of hydrogen bonding.

### B. Extrusion Process and Estimation of Residence Time

The residence time was dominated by the screw speed and feeding rates, which varied from minutes to hours. Additionally, the residence time can affect the formation of the cocrystals and affect the quality, morphology, and other characteristics of the products.

Three extrusion conditions were tried at the same temperature settings where feeding-screw speed was set at 6 g/min-50 rpm, 9 g/min-75 rpm, and 18 g/min-150 rpm. The residence time of each condition was 3.78 min, 2.80 min, and 1.22 min, and output 4.98 g/min, 8.31 g/min, and 17.52 g/min, respectively.

The process parameters and results are listed in Table 5. The extrusion of the IBU-NTM combinations is smooth where the torque is around 5.0 N/m (2.5% of maximum torque loading), which might be because of the complete melting of IBU and NTM in the mixing zone. The higher rpm results in a shorter residence time. The yield of the continuous extrusion can be as high as ˜1 kg/h. However, it can be noticed that a lower feeding rate was used whereas the screw speed was not set at its higher rpm, which means that there's room for further process optimization in order to achieve a much higher yield.

TABLE 5
Process results of extrusion of IBU-
NTM cocrystals at different conditions.

| Batch | 6 g/min-50 rpm | | 9 g/min-75 rpm | | 18 g/min-150 rpm | |
|---|---|---|---|---|---|---|
| Torque | 5.0 | N/m | 5.0 | N/m | 4.9 | N/m |
| Residence | 3 min 47 s | | 2 min 48 s | | 1 min 12 s | |
| time | | | | | | |
| Output | 0.2988 | kg/h | 0.4968 | kg/h | 1.0512 | kg/h |
| D50 | 123 | μm | 117 | μm | 98 | μm |
| AOR | 23.5° | | 24.3° | | 24.9° | |
| Tap density | 53.73 | g/ml | 58.06 | g/ml | 62.07 | g/ml |

The IBU-NTM cocrystals obtained from the extrusion process are mainly granules rather than big agglomerates or lumps (FIG. 21), where the D50 of granules decreased when increasing the screw speed. This might be because of the higher mechanical force or workloads to the downsizing of the granules.

The flowability of the cocrystals was identical, where the screw speed showed an insignificant effect on the AOR. The tap density of the cocrystals depended on the particle sizes, which are significantly affected by the screw speed.

The 6 g/min-50 rpm batch was repeated three times for reproducibility assessment . As shown in Table 6, the continuous extrusion of the cocrystals process showed adequate reproducibility, where most of the three batches' variation was within 5% variations. The variation of D50 is 12.62% which is still within an acceptable range for oral dosage forms, and this might be due to the fact that not only the process condition but also the sample collection and measuring procedure will affect the results.

TABLE 6
Reproducibility study of the continuous cocrystal extrusion process.

| 6 g/min-50 rpm | Batch 1 | | Batch 2 | | Batch 3 | | Average | | Variation |
|---|---|---|---|---|---|---|---|---|---|
| Residence | 3 min 42 s | | 3 min 53 s | | 3 min 46 s | | 3 min 47 s | | 2.45% |
| time | | | | | | | | | |
| Output | 0.2802 | kg/h | 0.3055 | kg/h | 0.3107 | kg/h | 0.2988 | kg/h | 5.46% |
| D50 | 138 | μm | 124 | μm | 107 | μm | 123 | μm | 12.62% |

| AOR | 23.4° | 23.4° | 23.6° | 23.5° | | 0.49% |
|---|---|---|---|---|---|---|
| Tap density | 52.98 | 54.07 | 54.14 | 53.73 | g/ml | 1.21 |

### C. Lenalidomide-Saccharin

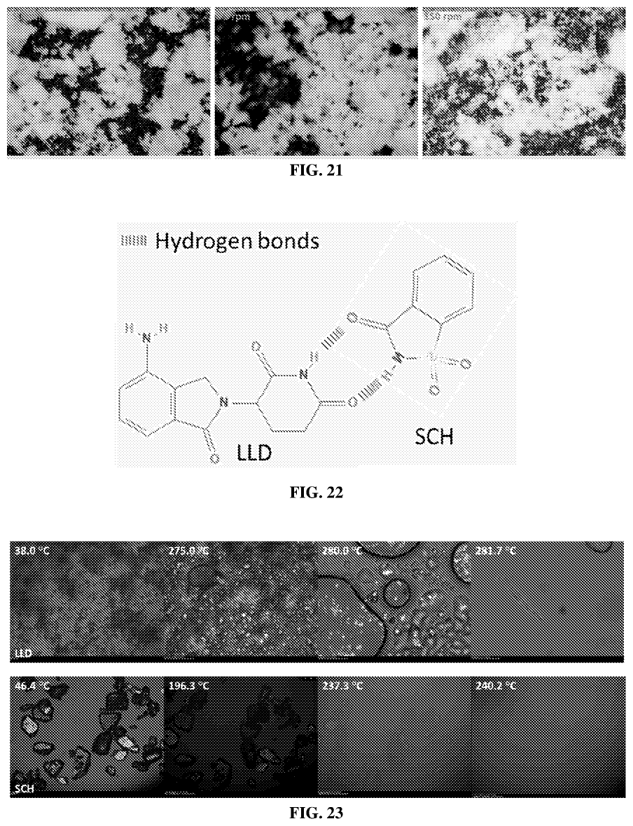

As shown in FIG. 22 above, the LLD and SCH can form cocrystals via forming hydrogen bonds between the amine and carboxyl groups. The extrusion of the LLD-SCH combinations is smooth, where the torque is around 5.8 N/m (2.9% of maximum torque loading).

i. Identification

PLM figures (FIG. 23) showed that LLD and SCH melt around 275.0° C. and 237.3° C., respectively. The physical mixture of the LLD-SCH starts melting around 207.1° C., while cocrystals start to melt around 157.1° C.

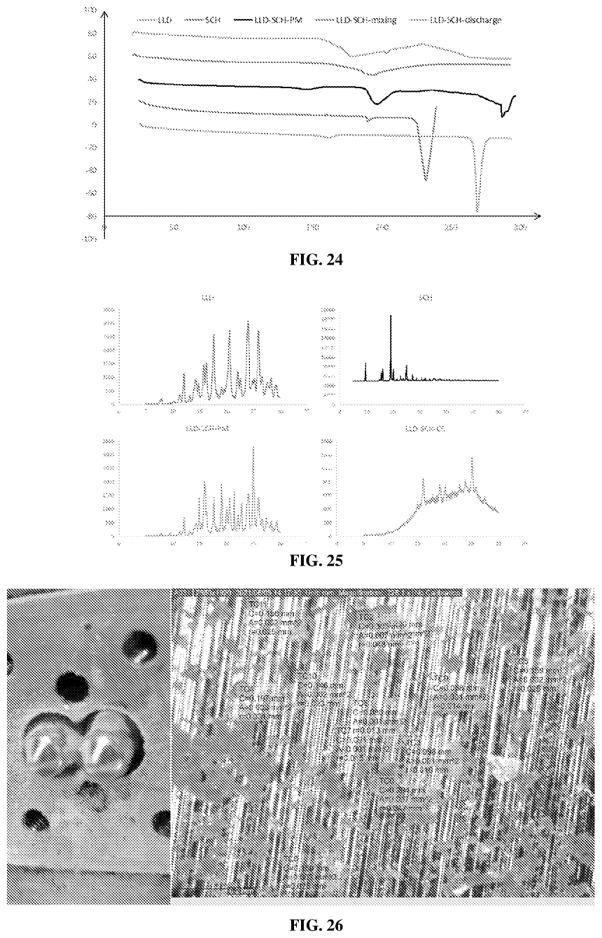

DSC figures (FIG. 24) showed LLD and SCH has a melting peak around 269.13° C. and 231.97° C., respectively. And two isolated melting peak can be observed in the physical mixture curve. The cocrystals curve showed a broaden melting peak of 178.58° C.

PXRD (FIG. 25) also proved the formation of the LLD-SCH cocrystals where the LLD showed characteristic peaks around 2θ of 16.24, 17.62, 20.60, 24.02, and 25.98°, and SCH showed peaks around 2θ of 9.68.,15.60, 16.12, 19.28, 25.22, and 27.50° . The cocrystals showed characteristic peaks around 2θ of 16.12, 19.14, 20.20, 22.90, 23.94, and 25.16°, 2°, which exhibited new diffractogram patterns compared to the LLD, SCH and their physical mixtures. The characteristic peaks of LLD and SCH absent in the cocrystals spectrum, which indicates the formation of new crystalline phase (Song et al., 2015; Song et al., 2014; Aitipamula et al., 2006; Almarsson et al., 2004).

ii. Process Qualities

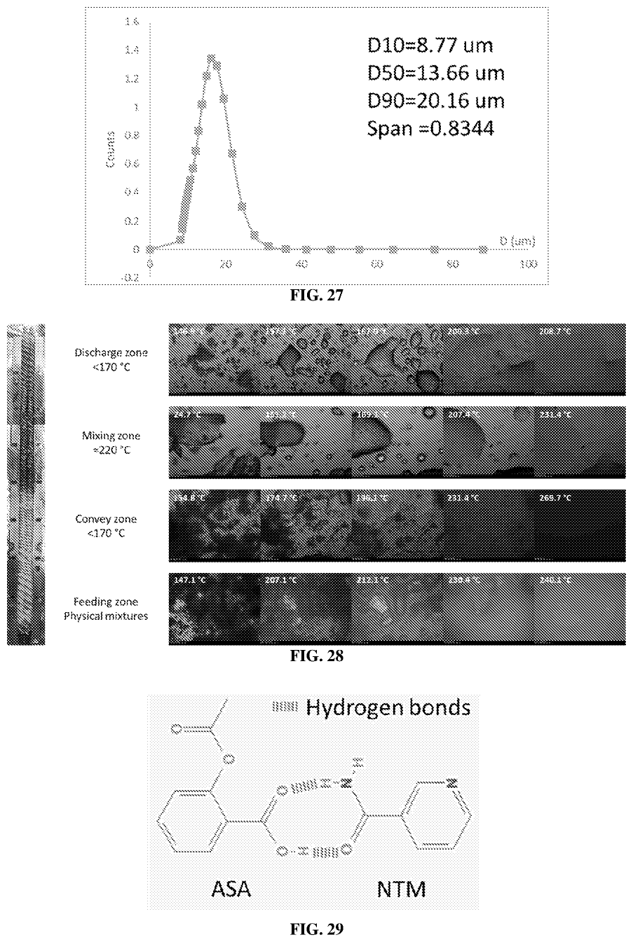

The particle size of LLD-SCH extrudates were smaller than other cocrystal formulations, where the D50 of the cocrystals is around 13 μm (FIG. 26). Additionally, the particle size distribution is adequate (Span=0.834), indicating that the cocrystal size is more uniform than other extruded cocrystals listed in the Table 3 (FIG. 27).

iii. Process Dynamics

As shown in the FIG. 28, the LLD and SCH were fed into the barrel in crystalline form from zone 1 and then slowly heated up. At zone 5, the physical mixtures melt and mix, then get conveyed to cooling and vent zones. At the end of zone 8, the molten materials started to recrystallize and discharged as cocrystals. As shown in the PLM graphs in FIG. 28 (right), the melting points of specimens collected from each zone decreased from the feeding to the end.

### D. Acetylsalicylic Acid-Nicotinamide

As shown in FIG. 29 above, the ASA and NTM can form cocrystals via forming hydrogen bonds between the amine and carboxyl groups. The extrusion of the ASA-NTM combinations is smooth, where the torque is around 4.0 N/m (2.0% of maximum torque loading). The extruded materials were not completely solidified as mentioned in table 1. Lower temperatures in zone 7 and 8 were tried, and the materials could be solidified. However, the extrudates were condensed and generated higher torques which can not be discharged.

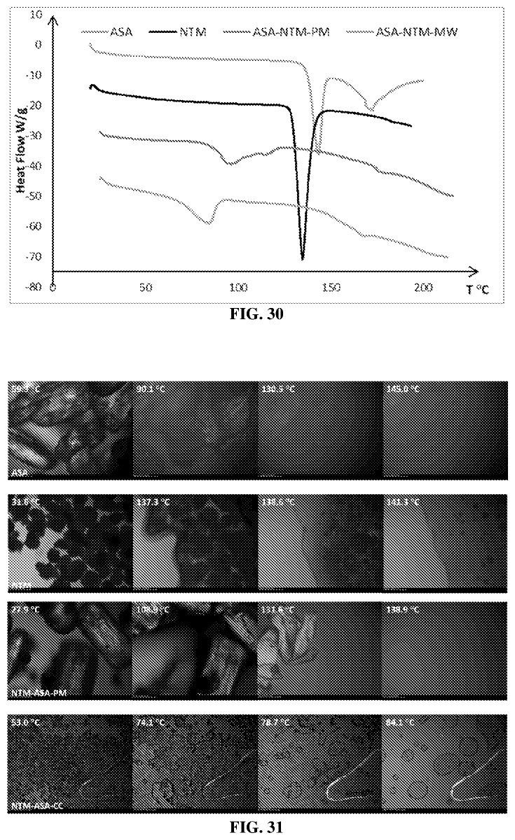

As shown in the FIG. 30 and FIG. 31, ASA has a reported melting point of the 135° C., while the grade we tested showed a melting at 143.6° C. NTM has a reported melting point of 130° C., and we tested at 134.6° C. The ASA-NTM physical mixtures showed adequate miscibility of the two ingredients, where the PM showed an attenuated melting endothermal peak with an onset of 94.0° C. Additionally, the PLM picture also confirmed that the melting of PM starts at about 100° C. and will completely melt around 132° C. The extrudates, cocrystal of ASA-NTM, showed a melting point at 84.1° C. in DSC analysis.

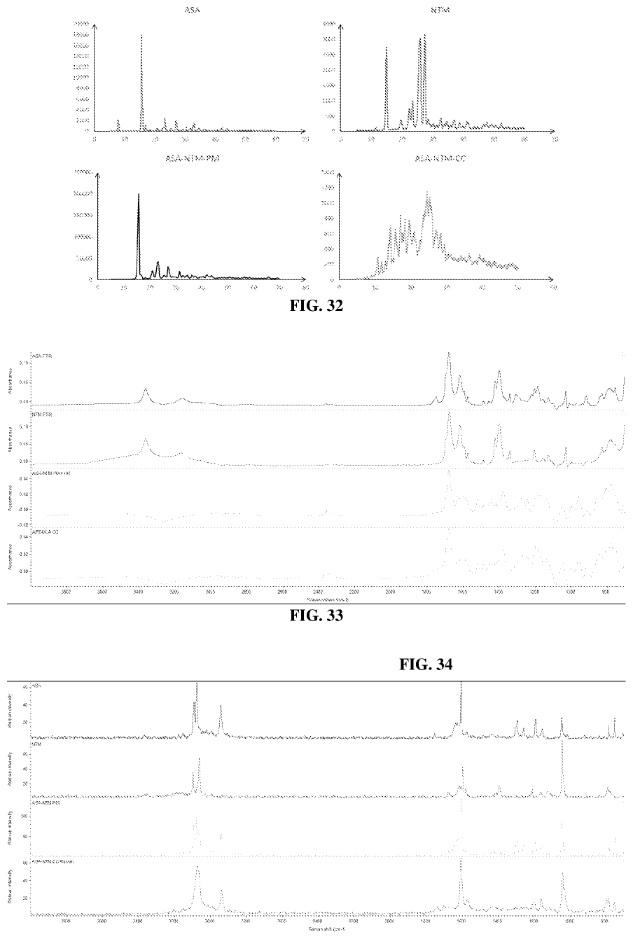

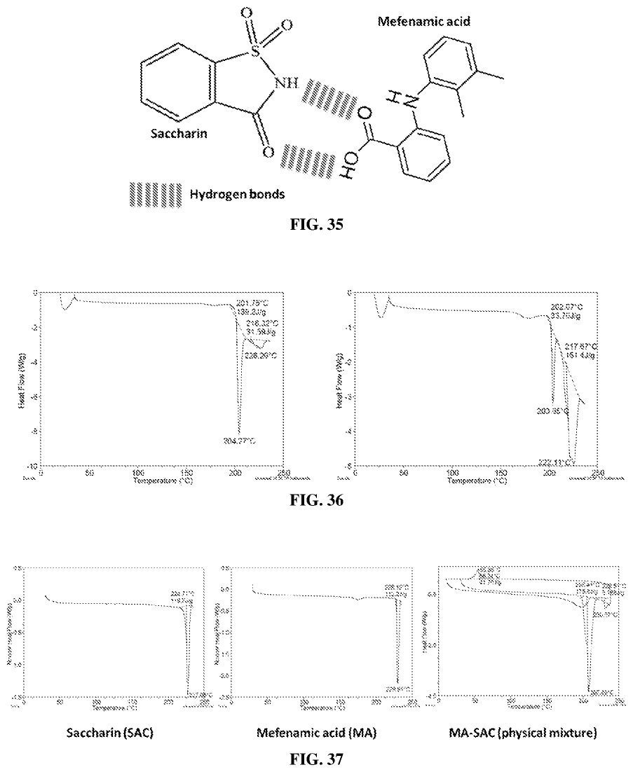

PXRD (FIG. 32) also confirmed the formation of the ASA-NTM cocrystals where the ASA showed characteristic peaks at around 20 of 7.84, 15.60, 23.32, 27.14, and 33.12°, and NTM showed peaks at around 20 of 15.00, 22.30, 23.12, and 27.50°.

The cocrystals showed peaks at around 20 of 14.36, 17.16, 18.40, 19.60, 24.56, and 25.32°, which exhibited new diffractogram patterns compared to the ASA, NTM and their physical mixtures. The characteristic peaks of ASA and NTM absent in the cocrystals spectrum, which indicates the formation of new crystalline phase (Almarsson et al., 2004; Hathwar et al., 2010; Dutt et al., 2020; Haeria & Ismail, 2015).

FTIR (FIG. 33) showed the intermolecular interactions of the cocrystals. The carboxyl group of ASA can be identified around wavenumbers of 1715 and 930 cm⁻¹, while the amine group of NTM can be identified around the wave number range of 1540-1450 cm⁻¹. The existence of the hydrogen bond in cocrystals results in the shift of the abovementioned peaks to the lower wavenumbers.

Raman (FIG. 34) showed the intramolecular movements of the cocrystals. The rotation and stretching movement of amine and carboxyl groups can be identified. The N—H and O—H frequencies of cocrystals were broadened and shifted to the lower wavenumbers sides because of hydrogen bonding.

### E. Mefenamic Acid-Saccharin

i. Background for the Molecule of Interest:

Mefenamic acid (Right): Molecular Weight (241.28 g/mol), Melting point (230-231° C.), Log P (5.12), pKa (4.2) Saccharin (Left): Molecular Weight (183.19 g/mol), Melting point (228° C.), Log P (0.91), pKa (1.31)

TABLE 7
Preformulation studies:

Solvent

| Compound | Me—OH | Et—OH | ET-Acetate | ACN |
|---|---|---|---|---|
| MA | No | Yes | Yes | No |
| SAC | Yes | No | Yes | Yes |
| SOL | Yes | Yes | Yes | Yes |
| VA64 | Yes | Yes | Yes | Yes |

Both the drug and conformer were soluble in ethyl acetate. Ethyl acetate was used to prepare pure MA-SAC cocrystals using slow solvent evaporation. Briefly, 1:1 molar ratio of MA and SAC were mixed using a mortar and pestle. This blend was dissolved in ethyl acetate at 50° C. in a glass beaker using a magnetic stirrer-hotplate. Once the physical mixture was dissolved, the solvent was placed on an ice bath to induce nucleation of the cocrystals, the beaker was then placed in fume hood until all the solvent was evaporated. After the solvent was evaporated, the crystals were placed in an oven at 60° C. to remove any residual solvent. The resultant crystals were tested for modulated differential scanning calorimetry. A clear phase separation was observed where crystals with two different habits were developed. Samples for each phase were collected and exposed to thermal analysis. DSC confirmed partial crystallization as seen in FIG. 36.

In order to determine the processing temperatures for thermal solvent processes such as HME, the physical mixture was exposed to a heat cool heat cycle using differential scanning calorimetry.

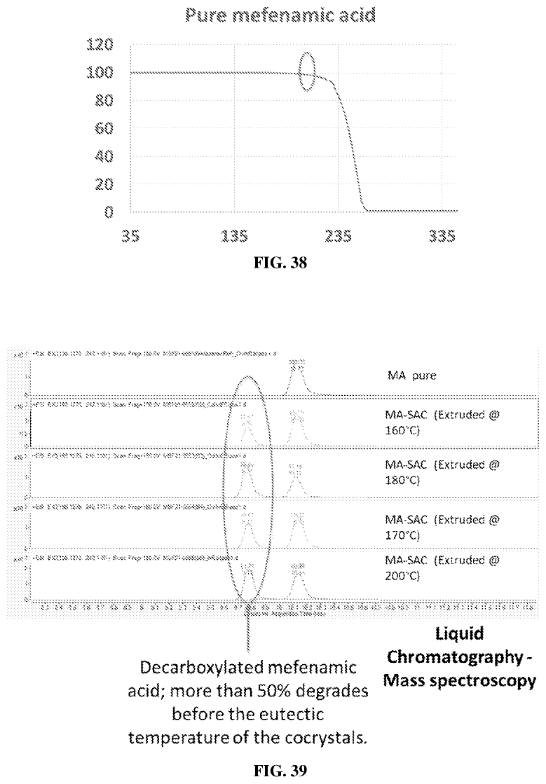

DSC of the physical mixture often indicates the eutectic point (a temperature at with a particular eutectic mixture melts together) and the second heating cycle depicts the melting point of the cocrystals. From the heat-cool-heat DSC the eutectic point for MA and SAC physical mixture is 207° C., whereas the melting points of MA and SAC are 229° C. and 227° C., respectively. The second heating cycle shows the melting point of the cocrystals, which is close to 200° C. However, the thermogravimetric analysis of MA suggests that the drug undergoes thermal degradation above 180° C. as seen in FIG. 38. This degradant was identified using high performance liquid chromatography with a mass spectrophotometer when mefenamic acid was processed using hot melt extrusion with saccharin at 200° C., 180° C., 170° C. and 160° C. as seen in FIG. 39.

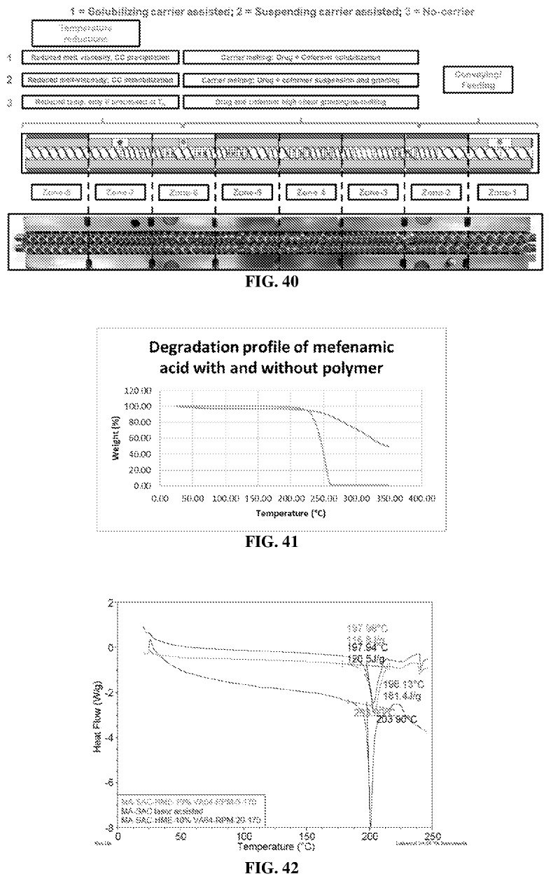

This reveals two problems with mefenamic acid, first is the phase separation during slow solvent evaporation, and second is the thermal decomposition on hot melt extrusion of neat MA-SAC physical mixture. These problems can be resolved by employing strategy-1 in the FIG. 40.

As per strategy 1, a solubilizing carrier i.e., Kollidon® VA64 was employed for manufacturing MA-SAC cocrystals. Mefenamic acid represents drugs which degrade below their eutectic temperature and melting temperature and have solubility in limited solvents leading to phase separation. In this example this problem was solved by using a polymer as a carrier with a glass transition temperature below the drug of interest, and which has the capacity of solubilizing the drug in its glassy state.

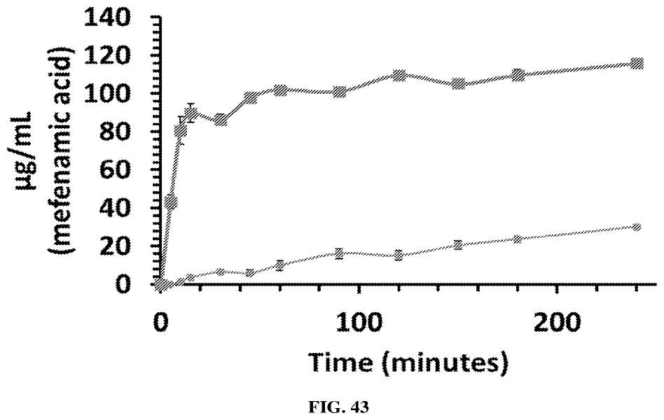

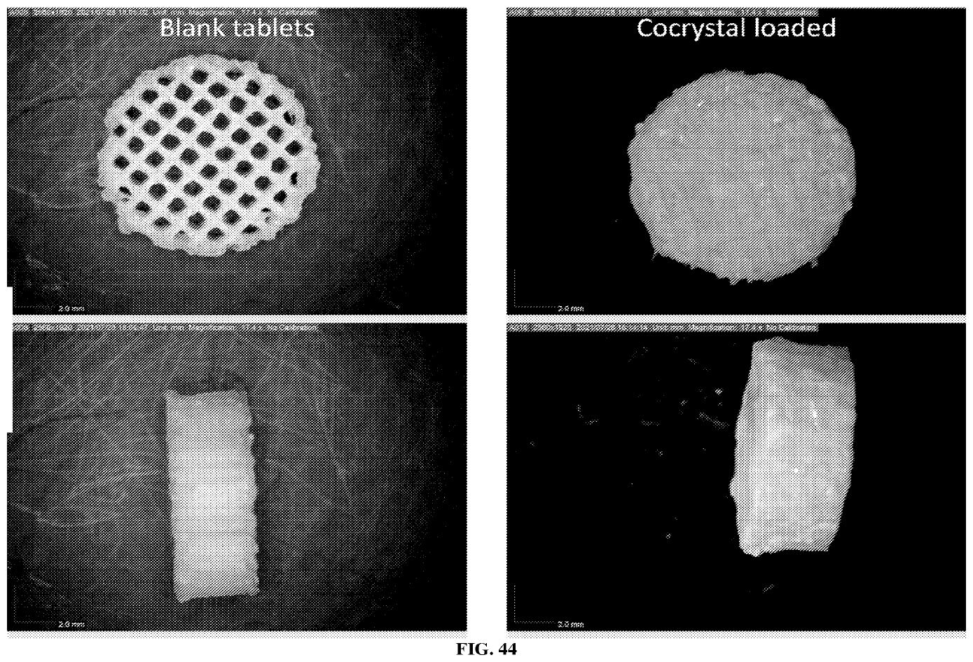

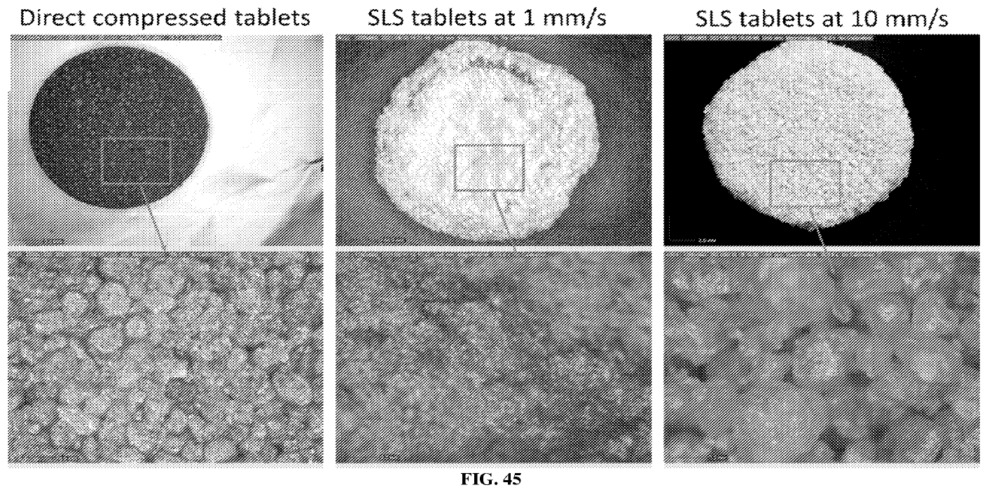

Example 4: Mefenamic Acid-Saccharin cocrystals were prepared with a polymeric carrier i.e., Kollidon® VA64. Where the composition was as follows: 10% Kollidon® VA64+90% MA-SAC (1:1 molar ratio) physical mixture. (Polymer range 5-30%, over 30% will give amorphous solid dispersions), the processing temperature was maintained below the melting point, eutectic point, and degradation temperature of both the drug and the conformer. This example represents the mechanism ‘1’ of the schematic depicted in FIG. 40. The extruder RPM was tested at 5 RPM and 20 RPM for this pair. Torque is required to be maintained between 20-70% using a combination of feed rate, screw design and RPM. The produced cocrystals were suitable for powder bed-based 3D printing applications and the cocrystals observed a four fold increase in the solubility during in vitro performance testing at pH 6.8. See, also, FIGS. 41-43.

### Demonstration of the AM Tablets with Cocrystals

As shown in the FIG. 44, the 3D FDM printed tablets with IBU-NTM cocrystals loaded were successfully made.

TABLE 8
Drug loading measurements and quality studies.

| | T_{Blank} | T_{Cocrystal Loaded} | Drug Loading | Drug Ratio |
|---|---|---|---|---|
| | (mg) | (mg) | (mg) | (w/w %) |
| 1 | 114 | 314 | 200 | 63.69 |
| 2 | 117 | 327 | 210 | 64.22 |
| 3 | 116 | 299 | 183 | 61.20 |
| Average | 115.67 | 313.33 | 197.67 | 63.04 |
| S.D. | 1.53 | 14.01 | 13.65 | 1.61 |
| Variation % | 1.32 | 4.47 | 6.91 | 2.56 |

As shown in Table 8, the 3D FDM printed tablets with cocrystal loaded showed adequate qualities, where the variation of each tablet is small (<6.91%). The drug loading can reach ˜64% with the current design.

As shown in FIG. 45, the IBU-NTM cocrystal-loaded tablets were successfully prepared via SLS printing. The directly compressed tablets showed that all the particles were physically compressed, while the 10 mm/s SLS tablets showed that Soluplus were slightly softened and sintered together and form the tablets. The 1 mm/s SLS tablets showed that all the particles were sintered together and formed the tablets.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.
Aitipamula et al., J. Ind. Inst. Sci., 97:227-243, 2017.
Almarsson et al., Chem. Comm., 1889-1896, 2004.
Dutt et al., Fut. J. Pharm. Sci., 6(32), 2020.
Haeria and Ismail, Int. J. Pharm. Res., 8(10):166-170, 2015.
Hathwar et al., Crystl. Growth Des., 10:3306-3310, 2010.
Song et al., Crystl. Growth Des., 15(10):4869-4875, 2015.
Song et al., Crystl. Growth Des., 14(6):3069-3077, 2014.

What is claimed is:

1. A composition comprising a co-crystal, wherein the co-crystal comprises
(A) an active pharmaceutical ingredient; and
(B) a co-former;
wherein the active pharmaceutical ingredient is a chemotherapeutic agent, an antibiotic, an antiviral agent, afenamic acid derivatives, lenalidomide, fleroxacin, or wherein the active pharmaceutical ingredient is ibuprofen and the co-former is saccharin, the active pharmaceutical ingredient is acetylsalicylic acid and the co-former is saccharin or the active pharmaceutical ingredient is carbamazepine and the co-former is maleic acid.

2. The composition of claim 1, wherein the composition comprises at least 50% of the active pharmaceutical ingredient and the co-former as a cocrystal.

3. The composition of claim 2, wherein the composition comprises at least 75% of the active pharmaceutical ingredient and the co-former as a cocrystal.

4. The composition of claim 3, wherein the composition comprises at least 95% of the active pharmaceutical ingredient and the co-former as a cocrystal.

5. The composition of claim 4, wherein the composition comprises at least 99% of the active pharmaceutical ingredient and the co-former as a cocrystal.

6. The composition according to any one of claims 1-5, wherein the active pharmaceutical ingredient is a BCS Class II drug.

7. The composition according to any one of claims 1-5, wherein the active pharmaceutical ingredient is a BCS Class IV drug.

8. The composition according to any one of claims 1-7, wherein the active pharmaceutical ingredient is an active pharmaceutical ingredient with a melting point of less than 250° C.

9. The composition of claim 8, wherein the melting point is less than 200° C.

10. The composition according to any one of claims 1-9, wherein the cocrystal has a melting point of less than 250° C.

11. The composition of claim 10, wherein the co-crystal melting point is less than 200° C.

12. The composition according to any one of claims 1-11, wherein the active pharmaceutical ingredient is a chemotherapeutic agent.

13. The composition of claim 12, wherein the chemotherapeutic agent is lenalidomide.

14. The composition according to any one of claims 1-11, wherein the active pharmaceutical ingredient is the antibiotic.

15. The composition of claim 14, wherein the antibiotic is fleroxacin.

16. The composition according to any one of claims 1-11, wherein the active pharmaceutical ingredient is a fenamic acid derivative.

17. The composition of claim 16, wherein the fenamic acid derivative is mefenamic acid.

18. The composition according to any one of claims 1-11, wherein the active pharmaceutical ingredient is a nonsteroidal anti-inflammatory.

19. The composition of claim 18, wherein the nonsteroidal anti-inflammatory is mefenamic acid, ibuprofen, or acetylsalicylic acid.

20. The composition according to any one of claims 1-19, wherein the co-former interacts with the active pharmaceutical ingredient through one or more non-covalent interactions.

21. The composition of claim 20, wherein the non-covalent interactions are ionic interactions, hydrogen bonding, halogen bonding, van der Waals forces, π-π interactions, or hydrophobic effects.

22. The composition according to any one of claims 1-21, wherein the co-former and the active pharmaceutical ingredient interact with two or more non-covalent interactions.

23. The composition according to any one of claims 1-22, wherein the co-former is a compound which modifies the solubility of the active pharmaceutical ingredient.

24. The composition of claim 23, wherein the co-former is a compound which is sparingly soluble and modifies the solubility of the active pharmaceutical ingredient.

25. The composition of claim 23, wherein the co-former is a compound which is sensitive to the environment and modifies the solubility of the active pharmaceutical ingredient.

26. The composition of claim 25, wherein the compound is sensitive to the pH of the environment.

27. The composition of claim 25, wherein the compound is sensitive to the temperature of the environment.

28. The composition according to any one of claims 1-27, wherein the co-former is a compound that has no therapeutic effect.

29. The composition according to any one of claims 1-27, wherein the co-former is a second active pharmaceutical ingredient.

30. The composition of claim 29, wherein the second active pharmaceutical ingredient is for the same disease or disorder as the first active pharmaceutical ingredient.

31. The composition of claim 29, wherein the second active pharmaceutical ingredient is for a different disease or disorder as the first active pharmaceutical ingredient.

32. The composition according to any one of claims 1-31, wherein the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio

33. The composition of claim 32, wherein the functional group is a NH₂, OH, C(O), C(O)OH, SH, or a nitrogen containing heterocycle.

34. The composition of claim 33, wherein the functional group is a nitrogen containing heterocycle, NH₂, OH, or SH.

35. The composition according to any one of claims 1-34, wherein the co-former is a flavoring compound.

36. The composition of claim 35, wherein the flavoring compound is saccharin.

37. The composition according to any one of claims 1-34, wherein the co-former is a carboxylic acid.

38. The composition of claim 37, wherein the co-former is maleic acid.

39. The composition according to any one of claims 1-34, wherein the co-former is a vitamin or a vitamin derivative.

40. The composition of claim 39, wherein the co-former is nicotinamide.

41. The composition according to any one of claims 1-34, wherein the co-former is a second active pharmaceutical ingredient.

42. The composition according to any one of claims 1-41, wherein the active pharmaceutical ingredient is ibuprofen and the co-former is saccharin.

43. The composition according to any one of claims 1-41, wherein the active pharmaceutical ingredient is acetylsalicylic acid and the co-former is saccharin.

44. The composition according to any one of claims 1-41, wherein the active pharmaceutical ingredient is carbamazepine and the co-former is maleic acid.

45. The composition according to any one of claim 1-44, wherein the pKₐ of the active pharmaceutical ingredient and the pKₐ of the co-former have a pKₐ difference of less than 3.

46. The composition of claim 45, wherein the pKₐ difference is less than 2.

47. The composition of claim 46, wherein the pKₐ difference is less than 1.

48. The composition according to any one of claims 1-47, wherein the physical mixture further comprises an excipient.

49. The composition of claim 48, wherein the excipient is a pharmaceutically acceptable thermoplastic polymer.

50. The composition of claim 49, wherein the active pharmaceutical ingredient or the co-former is not soluble in the pharmaceutically acceptable thermoplastic polymer.

51. The composition of claim 50, wherein the active pharmaceutical ingredient and the co-former are not soluble in the pharmaceutically acceptable thermoplastic polymer.

52. The composition according to any one of claims 1-51, wherein the composition comprises a molar ratio of the active pharmaceutical ingredient and the co-former from about 0.1 to about 10.

53. The composition of claim 52, wherein the molar ratio is from about 0.2 to about 5.

54. The composition of claim 53, wherein the molar ratio is from about 0.5 to about 2.

55. The composition of claim 54, wherein the molar ratio is about 1.

56. The composition of claim 54, wherein the molar ratio is about 2.

57. The composition according to any one of claims 1-56, wherein the composition is deposited onto a medical device.

58. The composition of claim 57, wherein the medical device is a tablet.

59. The composition of claim 58, wherein the tablet comprises an infill density from about 10% to about 90%.

60. The composition of claim 59, wherein the infill density is from about 20% to about 70%.

61. The composition of claim 60, wherein the infill density is from about 30% to about 50%.

62. The composition of claim 61, wherein the infill density is 30% or 50%.

63. The composition according to any one of claims 1-62, wherein the composition has been recrystallized onto the medical device.

64. A method of preparing a composition comprising a co-crystal comprising:
(A) obtaining an active pharmaceutical ingredient and a co-former to obtain a physical mixture;
(B) subjecting the physical mixture to an extrusion process to obtain a composition comprising a co-crystal;
wherein the extrusion process comprises two or more temperature zones.

65. The method of claim 64, wherein the composition comprises at least 50% of the active pharmaceutical ingredient and the co-former as a cocrystal.

66. The method of claim 65, wherein the composition comprises at least 75% of the active pharmaceutical ingredient and the co-former as a cocrystal.

67. The method of claim 66, wherein the composition comprises at least 95% of the active pharmaceutical ingredient and the co-former as a cocrystal.

68. The method of claim 67, wherein the composition comprises at least 99% of the active pharmaceutical ingredient and the co-former as a cocrystal.

69. The method according to any one of claims 64-68, wherein the active pharmaceutical ingredient is a BCS Class II drug.

70. The method according to any one of claims 64-68, wherein the active pharmaceutical ingredient is a BCS Class IV drug.

71. The method according to any one of claims 64-70, wherein the active pharmaceutical ingredient is an active pharmaceutical ingredient with a melting point of less than 250° C.

72. The method of claim 71, wherein the melting point is less than 200° C.

73. The method of claim 72, wherein the cocrystal has a melting point of less than 250° C.

74. The method of claim 73, wherein the co-crystal melting point is less than 200° C.

75. The method according to any one of claims 64-74, wherein the active pharmaceutical ingredient is a chemotherapeutic agent.

76. The method of claim 69, wherein the chemotherapeutic agent is lenalidomide.

77. The method according to any one of claims 64-74, wherein the active pharmaceutical ingredient is the antibiotic.

78. The method of claim 77, wherein the antibiotic is fleroxacin.

79. The method according to any one of claims 64-74, wherein the active pharmaceutical ingredient is a fenamic acid derivative.

80. The method of claim 79, wherein the fenamic acid derivative is mefenamic acid.

81. The method according to any one of claims 64-74, wherein the active pharmaceutical ingredient is a nonsteroidal anti-inflammatory.

82. The method of claim 81, wherein the nonsteroidal anti-inflammatory is mefenamic acid, ibuprofen, or acetylsalicylic acid.

83. The method according to any one of claims 64-82, wherein the co-former interacts with the active pharmaceutical ingredient through one or more non-covalent interactions.

84. The method of claim 83, wherein the non-covalent interactions are ionic interactions, hydrogen bonding, halogen bonding, van der Waals forces, π-π interactions, or hydrophobic effects.

85. The method according to any one of claims 64-84, wherein the co-former and the active pharmaceutical ingredient interact with two or more non-covalent interactions.

86. The method according to any one of claims 64-85, wherein the co-former is a compound which modifies the solubility of the active pharmaceutical ingredient.

87. The method of claim 86, wherein the co-former is a compound which is sparingly soluble and modifies the solubility of the active pharmaceutical ingredient.

88. The method of claim 86, wherein the co-former is a compound which is sensitive to the environment and modifies the solubility of the active pharmaceutical ingredient.

89. The method of claim 88, wherein the compound is sensitive to the pH of the environment.

90. The method of claim 89, wherein the compound is sensitive to the temperature of the environment.

91. The method according to any one of claims 64-91, wherein the co-former is a compound that has no therapeutic effect.

92. The method according to any one of claims 64-91, wherein the co-former is a second active pharmaceutical ingredient.

93. The method of claim 92, wherein the second active pharmaceutical ingredient is for the same disease or disorder as the first active pharmaceutical ingredient.

94. The method of claim 92, wherein the second active pharmaceutical ingredient is for a different disease or disorder as the first active pharmaceutical ingredient.

95. The method according to any one of claims 64-94, wherein the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio

96. The method of claim 95, wherein the functional group is a NH₂, OH, C(O), C(O)OH, SH, or a nitrogen containing heterocycle.

97. The method of claim 96, wherein the functional group is a nitrogen containing heterocycle, NH₂, OH, or SH.

98. The method according to any one of claims 64-95, wherein the co-former is a flavoring compound.

99. The method of claim 98, wherein the flavoring compound is saccharin.

100. The method according to any one of claims 64-97, wherein the co-former is a carboxylic acid.

101. The method of claim 100, wherein the co-former is maleic acid.

102. The method according to any one of claims 64-82, wherein the co-former is a vitamin or a vitamin derivative.

103. The method of claim 102, wherein the co-former is nicotinamide.

104. The method according to any one of claims 64-82, wherein the co-former is a second active pharmaceutical ingredient.

105. The method according to any one of claim 64-104, wherein the pKₐ of the active pharmaceutical ingredient and the pKₐ of the co-former have a pKₐ difference of less than 3.

106. The method of claim 105, wherein the pKₐ difference is less than 2.

107. The method of claim 106, wherein the pKₐ difference is less than 1.

108. The method according to any one of claims 64-107, wherein the physical mixture further comprises an excipient.

109. The method of claim 108, wherein the excipient is a pharmaceutically acceptable thermoplastic polymer.

110. The method of claim 109, wherein the highest temperature of any of the temperature zone is below the melting temperature of the pharmaceutically acceptable thermoplastic polymer

111. The method of claim 109, wherein the highest temperature of any of the temperature zone is at the melting temperature of the pharmaceutically acceptable thermoplastic polymer

112. The method of claim 109, wherein the highest temperature of any of the temperature zone is above the melting temperature of the pharmaceutically acceptable thermoplastic polymer

113. The method according to any one of claims 64-109, wherein the composition comprises a molar ratio of the active pharmaceutical ingredient and the co-former from about 0.1 to about 10.

114. The method of claim 113, wherein the molar ratio is from about 0.2 to about 5.

115. The method of claim 114, wherein the molar ratio is from about 0.5 to about 2.

116. The method of claim 115, wherein the molar ratio is about 1.

117. The method of claim 115, wherein the molar ratio is about 2.

118. The method according to any one of claims 64-117, wherein each of the two or more temperature zones are each at a distinct temperature.

119. The method according to any one of claims 64-118, wherein the extrusion process comprises two, three, four, five, six, seven, or eight temperature zones.

120. The method of claim 119, wherein the extrusion process comprises three, four, or five temperature zones.

121. The method of claim 120, wherein the extrusion process comprises four temperature zones.

122. The method according to any one of claims 64-121, wherein the first temperature zone has a temperature from about 30° C. to about 150° C.

123. The method of claim 122, wherein the first temperature zone is from about 50° C. to about 100° C.

124. The method of claim 123, wherein the first temperature zone is from about 60° C. to about 80° C.

125. The method of claim 124, wherein the first temperature zone is about 70° C.

126. The method according to any one of claims 64-125, wherein the second temperature zone has a temperature from about 50° C. to about 200° C.

127. The method of claim 126, wherein the second temperature zone is from about 75° C. to about 180° C.

128. The method of claim 127, wherein the second temperature zone is from about 100° C. to about 160° C.

129. The method of claim 128, wherein the second temperature zone is about 140° C.

130. The method according to any one of claims 64-129, wherein the third temperature zone has a temperature from about 75° C. to about 250° C.

131. The method of claim 130, wherein the third temperature zone is from about 100° C. to about 220° C.

132. The method of claim 131, wherein the third temperature zone is from about 140° C. to about 200° C.

133. The method of claim 132, wherein the third temperature zone is about 160° C.

134. The method according to any one of claims 64-133, wherein the fourth temperature zone has a temperature from about 75° C. to about 300° C.

135. The method of claim 134, wherein the fourth temperature zone is from about 100° C. to about 250° C.

136. The method of claim 135, wherein the fourth temperature zone is from about 150° C. to about 225° C.

137. The method of claim 136, wherein the fourth temperature zone is about 180° C.

138. The method according to any one of claims 64-137, wherein the ejection temperature is a temperature from about 75° C. to about 250° C.

139. The method of claim 138, wherein the ejection temperature is from about 100° C. to about 220° C.

140. The method of claim 139, wherein the ejection temperature is from about 140° C. to about 200° C.

141. The method of claim 140, wherein the ejection temperature is about 160° C.

142. The method according to any one of claims 64-141, wherein the melting point of the pharmaceutically acceptable thermoplastic polymer is below the melting point of the active pharmaceutical ingredient or co-former.

143. The method according to any one of claims 64-141, wherein the melting point of the pharmaceutically acceptable thermoplastic polymer is below the melting point of the active pharmaceutical ingredient and co-former.

144. The method according to any one of claims 64-143, wherein the extrusion process has a rotation speed from about 10 rpm to about 250 rpm.

145. The method of claim 144, wherein the rotation speed is from about 20 rpm to about 200 rpm.

146. The method of claim 145, wherein the rotation speed is from about 25 rpm to about 150 rpm.

147. The method of claim 146, wherein the rotation speed is about 50 rpm, 75 rpm, or 150 rpm.

148. The method according to any one of claims 64-147, wherein the extrusion process has a feed rate of the physical mixture from about 1 g/min to about 50 g/min.

149. The method of claim 148, wherein the feed rate is from about 1.5 g/min to about 50 g/min.

150. The method of claim 149, wherein the feed rate is from about 2 g/min to about 20 g/min.

151. The method of claim 150, wherein the feed rate is about 5 g/min.

152. A pharmaceutical composition prepared according to the methods described in any one of claims 64-151.

153. A method of preparing a pharmaceutical composition comprising:
(A) obtaining a composition according to any one of claim 1-63 or 152 or composition prepared according to any one of claims 64-151;
(B) subjecting the composition to an additive manufacturing technique to obtain a pharmaceutical composition;
wherein the pharmaceutical composition is prepared as a unit dose.

154. The method of claim 153, wherein the additive manufacturing technique is vat photopolymerization, material jetting, binder jetting, powder bed fusion, material extrusion, directed energy deposition, or sheet lamination.

155. The method of claim 154, wherein the additive manufacturing technique is a fused deposition modeling technique.

156. The method according to any one of claims 153-155, wherein the composition is present as a filament.

157. The method according to any one of claims 153-155, wherein the composition is present as a powder, granule, or a particle.

158. The method according to any one of claims 153-157, wherein the composition further comprises a pharmaceutically acceptable polymer.

159. The method according to any one of claims 153-158, wherein the composition is deposited onto a dosage form.

160. The method of claim 153, wherein the additive manufacturing technique is binder spraying.

161. The method of claim 153, wherein the additive manufacturing technique is selective laser sintering.

162. The method of either claim 153 or claim 154, wherein the unit dose is an oral dosage form.

163. The method of claim 162, wherein the oral dosage form is a tablet or capsule.

164. A method of preparing a drug-loaded medical device comprising:
(A) obtaining a composition according to any one of claim 1-63 or 152 or composition prepared according to any one of claims 64-151;
(B) dissolving the composition in a solvent to form a drug-containing solution; and
(C) placing an unloaded medical device into the drug-containing solution and allowing the composition to crystalize onto the medical device to form a drug-loaded medical device.

165. The method of claim 164, wherein the method comprises heating the drug-containing solution.

166. The method of claim 165, wherein the drug-containing solution is heated to a temperature from about 30° C. to about 150° C.

167. The method of claim 166, wherein the temperature is from about 40° C. to about 100 ° C.

168. The method of claim 167, wherein the temperature is from about 50° C. to about 80° C.

169. The method according to any one of claims 164-168, wherein the solvent is an organic solvent.

170. The method of claim 169, wherein the organic solvent is a C1-C8 alcohol.

171. The method of claim 170, wherein the organic solvent is ethanol.

172. The method according to any one of claims 164-171, wherein the medical device is a tablet.

173. The method of claim 172, wherein the table has an infill density from about 10% to about 90%.

174. The method of claim 173, wherein the infill density is from about 20% to about 70%.

175. The method of claim 174, wherein the infill density is from about 30% to about 50%.

176. The method of claim 175, wherein the infill density is 30% or 50%.

177. A method of treating or preventing a disease or disorder in a patient comprising administering to the patient in need thereof a therapeutically effective amount of a composition according to any one of claim 1-63 or 152 or a composition prepared according to the methods of any one of claim 64-151 or 153-176; wherein the active pharmaceutical ingredient is sufficient to treat or prevent the disease or disorder.
